(19) United States
(12) Patent Application Publication
(10) Pub. No.: US 2010/0113480 A1
(43) Pub. Date: May 6, 2010

(54) PROCESS FOR THE PREPARATION OF TRI-SUBSTITUTED PYRIDINE AND TRI-SUBSTITUTED PYRIMIDINE DERIVATIVES USEFUL AS GDIR AGONISTS

(76) Inventor: Michael Reuman, New Hope, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/608,015

(22) Filed: Oct. 29, 2009

Related U.S. Application Data
(60) Provisional application No. 61/109,714, filed on Oct. 30, 2008.

(51) Int. Cl. A61K 31/497; C07D 239/34; A61P 3/10

(52) U.S. Cl. 514/256; 544/298

(57) ABSTRACT

The present invention is directed to novel processes for the preparation of tri-substituted pyridine and tri-substituted pyrimidine derivatives, useful as glucose dependent insulinotropic receptor agonist, for the treatment of metabolic-related disorders and complications thereof, such as, diabetes and obesity.

### CROSS REFERENCE TO RELATED APPLICATION

This application claims the benefit of U.S. Provisional Application No. 61/109,714, filed Oct. 30, 2008, which is incorporated by reference herein.

### FIELD OF THE INVENTION

The present invention is directed to novel processes for the preparation of tri-substituted pyridine and tri-substituted pyrimidine derivatives, useful as glucose dependent insulinotropic receptor agonist, for the treatment of metabolic-related disorders and complications thereof, such as, diabetes and obesity.

### BACKGROUND OF THE INVENTION

Diabetes mellitus is a serious disease afflicting over 100 million people worldwide. In the United States, there are more than 12 million diabetics, with 600,000 new cases diagnosed each year.

Diabetes mellitus is a diagnostic term for a group of disorders characterized by abnormal glucose homeostasis resulting in elevated blood sugar. There are many types of diabetes, but the two most common are Type I (also referred to as insulin-dependent diabetes mellitus or IDDM) and Type II (also referred to as non-insulin-dependent diabetes mellitus or NIDDM). The etiology of the different types of diabetes is not the same; however, everyone with diabetes has two things in common: overproduction of glucose by the liver and little or no ability to move glucose out of the blood into the cells where it becomes the body's primary fuel. People who do not have diabetes rely on insulin, a hormone made in the pancreas, to move glucose from the blood into the cells of the body. However, people who have diabetes either don't produce insulin or can't efficiently use the insulin they produce; therefore, they can't move glucose into their cells. Glucose accumulates in the blood creating a condition called hyperglycemia, and over time, can cause serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

People with IDDM, which accounts for about 5% to 10% of those who have diabetes, don't produce insulin and therefore must inject insulin to keep their blood glucose levels normal. IDDM is characterized by low or undetectable levels of endogenous insulin production caused by destruction of the insulin-producing 13 cells of the pancreas, the characteristic that most readily distinguishes IDDM from NIDDM. IDDM, once termed juvenile-onset diabetes, strikes young and older adults alike.

Approximately 90 to 95% of people with diabetes have Type II (or NIDDM). NIDDM subjects produce insulin, but the cells in their bodies are insulin resistant: the cells don't respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production (Rotwein, R. et al. N. Engl. J. Med. 308, 65-71 (1983)). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious or even clinically unapparent, making diagnosis difficult.

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important contributors to the disease process (Rimoin, D. L., et. al. Emery and Rimoin's Principles and Practice of Medical Genetics 3^{rd} Ed. 1:1401-1402 (1996)).

Many people with NIDDM have sedentary lifestyles and are obese; they weigh approximately 20% more than the recommended weight for their height and build. Furthermore, obesity is characterized by hyperinsulinemia and insulin resistance, a feature shared with NIDDM, hypertension and atherosclerosis.

Obesity and diabetes are among the most common human health problems in industrialized societies. In industrialized countries a third of the population is at least 20% overweight. In the United States, the percentage of obese people has increased from 25% at the end of the 1970s, to 33% at the beginning the 1990s. Obesity is one of the most important risk factors for NIDDM. Definitions of obesity differ, but in general, a subject weighing at least 20% more than the recommended weight for his/her height and build is considered obese. The risk of developing NIDDM is tripled in subjects 30% overweight, and three-quarters with NIDDM are overweight.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increase insulin secretion balances insulin resistance and protects patients from hyperglycemia (Le Stunff, et al. Diabetes 43, 696-702 (1989)). However, after several decades, β cell function deteriorates and non-insulin-dependent diabetes develops in about 20% of the obese population (Pederson, P. Diab. Metab. Rev. 5, 505-509 (1989)) and (Brancati, F. L., et al., Arch. Intern. Med. 159, 957-963 (1999)). Given its high prevalence in modern societies, obesity has thus become the leading risk factor for NIDDM (Hill, J. O., et al., Science 280, 1371-1374 (1998)). However, the factors which predispose a fraction of patients to alteration of insulin secretion in response to fat accumulation remain unknown.

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see TABLE below). There are problems with this definition in that it does not take into account the proportion of body mass that is muscle in relation to fat (adipose tissue). To account for this, obesity can also be defined on the basis of body fat content: greater than 25% and 30% in males and females, respectively.

### Classification of Weight by Body Mass Index (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Compounds marketed as anti-obesity agents include Orlistat (XENICAL™) and Sibutramine. Orlistat (a lipase inhibitor) inhibits fat absorption directly and tends to produce a high incidence of unpleasant (though relatively harmless) side-effects such as diarrhea. Sibutramine (a mixed 5-HT/noradrenaline reuptake inhibitor) can increase blood pressure and heart rate in some patients. The serotonin releaser/reuptake inhibitors fenfluramine (Pondimin™) and dexfenfluramine (Redux™) have been reported to decrease food intake and body weight over a prolonged period (greater than 6 months). However, both products were withdrawn after reports of preliminary evidence of heart valve abnormalities associated with their use. Accordingly, there is a need for the development of a safer anti-obesity agent.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of these conditions based on the prevention of obesity (Perry, I. J., et al., BMJ 310, 560-564 (1995)).

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

Jones, R. M., et al., in PCT Publication WO2006/083491, published Oct. 8, 2006 disclose substituted pyridinyl and pyrimidinyl derivatives, compounds which bind to and modulate the activity of a GPCR and uses thereof.

### SUMMARY OF THE INVENTION

The present invention is directed to a process for the preparation of a compound of formula (I)

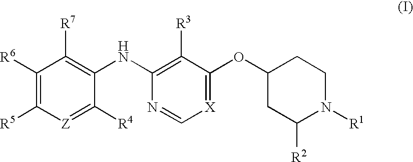

wherein:

X is N or CR⁸; wherein R⁸ is H or halogen;

Z is CH or N;

R¹ is carbo-C₁₋₆-alkoxy, oxadiazolyl or pyrimidinyl wherein said carbo-C₁₋₆-alkoxy, oxadiazolyl and pyrimidinyl are each optionally substituted with 1 or 2 substituents selected independently from the group consisting of C₁₋₄alkyl, C₁₋₄alkoxy and C₃₋₅cycloalkyl;

R² is H or C₁₋₄alkyl;

R³ is C₁₋₄ alkoxy, O—C₂₋₄-alkynyl or hydroxyl;

R⁴ is selected from the group consisting of H, C₁₋₄alkoxy, C₁₋₄alkyl, C₂₋₄alkynyl and halogen;

R⁵ is selected from the group consisting of C₁₋₄acylsulfonamide, C₁₋₄alkoxy, C₁₋₄alkyl, C₁₋₄alkylamino, C₁₋₄alkylsulfonyl, C₁₋₄alkylthio, cyano, heterocyclyl, di-C₁₋₄-dialkylamino and sulfonamide, wherein said C₁₋₄alkoxy, C₁₋₄alkyl, C₁₋₄alkylamino, C₁₋₄alkylsulfonyl, C₁₋₄alkylthio, di-C₁₋₄-dialkylamino and heterocyclyl are each optionally substituted with 1 or 2 substituents selected independently from the group consisting of C₂₋₄alkynyl, C₁₋₄alkoxy, C₁₋₄alkylcarboxamide, C₁₋₄alkylsulfonyl, C₃₋₅cycloalkyl, C₃₋₅cycloalkyloxy, di-C₁₋₄-alkylcarboxamide, hydroxyl and phosphonooxy, wherein said C₁₋₄alkylcarboxamide is optionally substituted with hydroxyl; or

or R⁵ is a group of Formula (A):

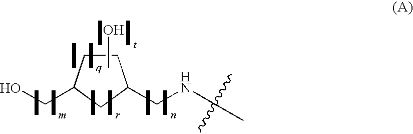

wherein “m”, “n” and “q” are each independently 0, 1, 2 or 3; “r” is 0, 1 or 2; and “t” is 0 or 1;

R⁶ is H or halogen;

R⁷ is H or C₁₋₄alkyl;

or a pharmaceutically acceptable salt, solvate or hydrate thereof; comprising

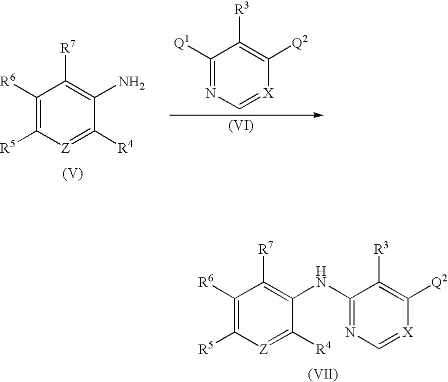

reacting a compound of formula (V) with a compound of formula (VI), wherein Q¹ and Q² are each independently a leaving group; in the presence of an acid catalyst; in a first organic solvent; to yield the corresponding compound of formula (VII);

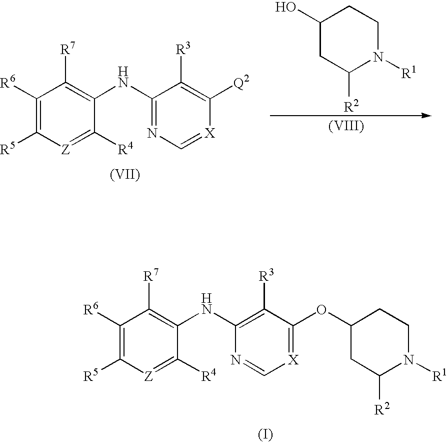

reacting the compound of formula (VII) with a compound of formula (VIII); in the presence of a base, which base is strong enough to de-protonate the hydroxy group which is bound at the 4-position of the piperidinyl portion of the compound of formula (VIII); in a third organic solvent; to yield the corresponding compound of formula (I).

The present invention is further directed to a process for the preparation of a compound of formula (I)

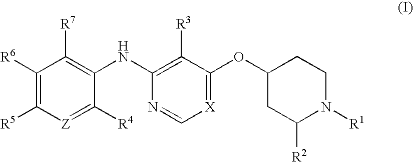

wherein:

X is N or CR⁸; wherein R⁸ is H or halogen;

Z is CH or N;

R¹ is carbo-C₁₋₆-alkoxy, oxadiazolyl or pyrimidinyl wherein said carbo-C₁₋₆-alkoxy, oxadiazolyl and pyrimidinyl are each optionally substituted with 1 or 2 substituents selected independently from the group consisting of C₁₋₄alkyl, C₁₋₄alkoxy and C₃₋₅cycloalkyl;

R² is H or C₁₋₄alkyl;

R³ is C₁₋₄ alkoxy, O—C₂₋₄-alkynyl or hydroxyl;

R⁴ is selected from the group consisting of H, C₁₋₄alkoxy, C₁₋₄alkyl, C₂₋₄alkynyl and halogen;

R⁵ is selected from the group consisting of C₁₋₄acylsulfonamide, C₁₋₄alkoxy, C₁₋₄alkyl, C₁₋₄alkylamino, C₁₋₄alkylsulfonyl, C₁₋₄alkylthio, cyano, heterocyclyl, di-C₁₋₄-dialkylamino and sulfonamide, wherein said C₁₋₄alkoxy, C₁₋₄alkyl, C₁₋₄alkylamino, C₁₋₄alkylsulfonyl, C₁₋₄alkylthio, di-C₁₋₄-dialkylamino and heterocyclyl are each optionally substituted with 1 or 2 substituents selected independently from the group consisting of C₂₋₄alkynyl, C₁₋₄alkoxy, C₁₋₄alkylcarboxamide, C₁₋₄alkylsulfonyl, C₃₋₅cycloalkyl, C₃₋₅cycloalkyloxy, di-C₁₋₄-alkylcarboxamide, hydroxyl and phosphonooxy, wherein said C₁₋₄alkylcarboxamide is optionally substituted with hydroxyl; or

or R⁵ is a group of Formula (A):

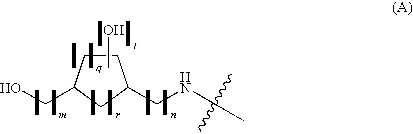

wherein “m”, “n” and “q” are each independently 0, 1, 2 or 3; “r” is 0, 1 or 2; and “t” is 0 or 1;

R⁶ is H or halogen;

R⁷ is H or C₁₋₄alkyl;

or a pharmaceutically acceptable salt, solvate or hydrate thereof; comprising

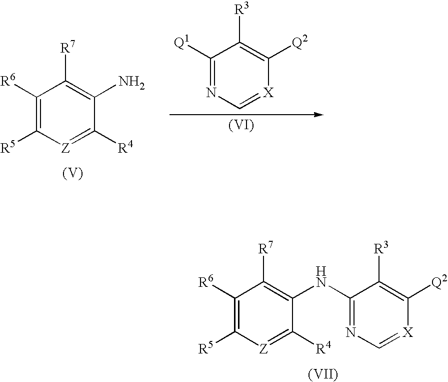

reacting a compound of formula (V) with a compound of formula (VI), wherein Q¹ and Q² are each independently a leaving group; in the presence of carbonate base; in a second organic solvent; to yield the corresponding compound of formula (VII):

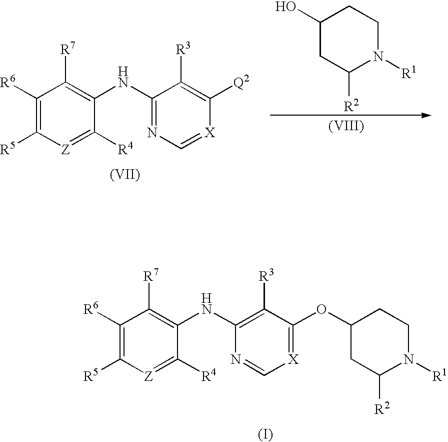

reacting the compound of formula (VII) with a compound of formula (VIII); in the presence of a base, which base is strong enough to de-protonate the hydroxy group which is bound at the 4-position of the piperidinyl portion of the compound of formula (VIII); in a third organic solvent; to yield the corresponding compound of formula (I).

In an embodiment, the present invention is directed to a process for the preparation of a compound of formula (I-S)

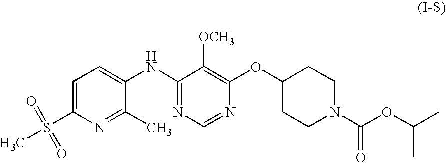

(also known as 4-[6-(6-methanesulfonyl-2-methyl-pyridin-3-ylamino)-5-methoxy-pyrimidin-4-yloxy]-piperidine-1-carboxylic acid isopropyl ester), or a pharmaceutically acceptable salt, solvate or hydrate thereof; comprising

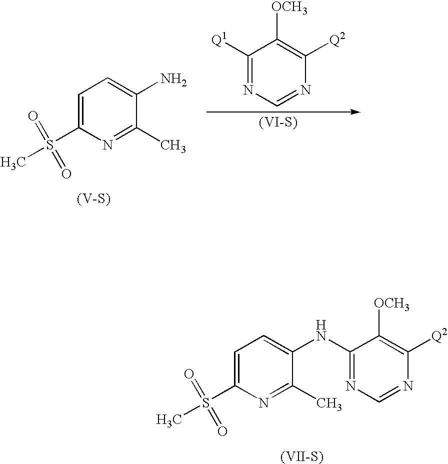

reacting a compound of formula (V-S) with a compound of formula (VI-S), wherein Q¹ and Q² are each independently a leaving group; in the presence of an acid catalyst; in a first organic solvent; to yield the corresponding compound of formula (VII-S);

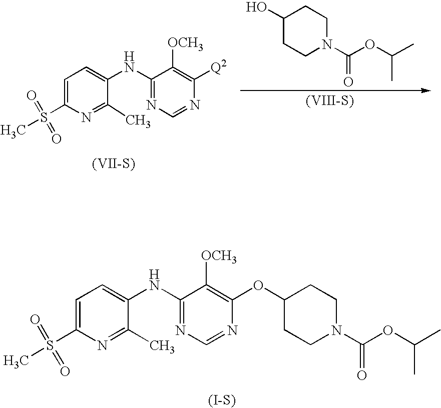

reacting the compound of formula (VII-S) with a compound of formula (VIII-S); in the presence of a base, which base is strong enough to de-protonate the hydroxy group which is bound at the 4-position of the piperidinyl portion of the compound of formula (VIII-S); in a third organic solvent; to yield the corresponding compound of formula (I-S).

The present invention is directed to a process for the preparation of a compound of formula (I-S)

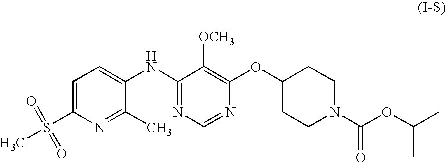

(also known as 4-[6-(6-methanesulfonyl-2-methyl-pyridin-3-ylamino)-5-methoxy-pyrimidin-4-yloxy]-piperidine-1-carboxylic acid isopropyl ester), or a pharmaceutically acceptable salt, solvate or hydrate thereof; comprising

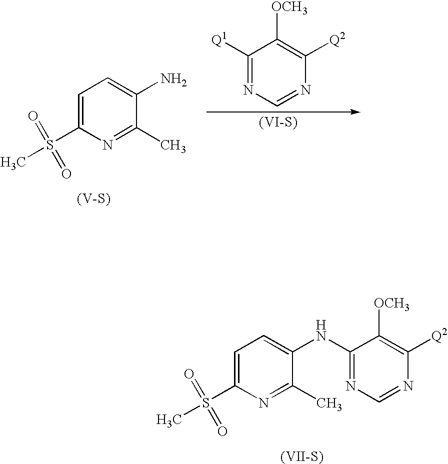

reacting a compound of formula (V-S) with a compound of formula (VI-S), wherein Q¹ and Q² are each independently a leaving group; in the presence of carbonate base; in a second organic solvent; to yield the corresponding compound of formula (VII-S);

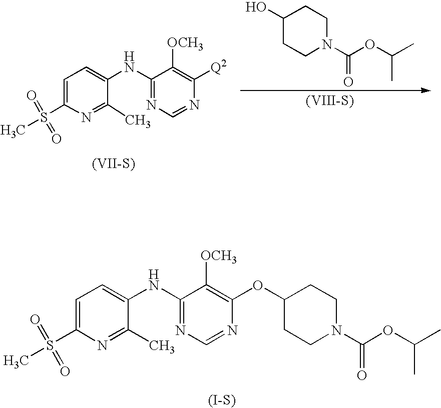

reacting the compound of formula (VII-S) with a compound of formula (VIII-S); in the presence of a base, which base is strong enough to de-protonate the hydroxy group which is bound at the 4-position of the piperidinyl portion of the compound of formula (VIII-S); in a third organic solvent; to yield the corresponding compound of formula (I-S).

The present invention is further directed to a product prepared according to any of processes described herein.

Illustrative of the invention is a pharmaceutical composition comprising a pharmaceutically acceptable carrier and a product prepared according to any of the processes described herein. An illustration of the invention is a pharmaceutical composition made by mixing a product prepared according to any of the processes described herein and a pharmaceutically acceptable carrier. Illustrating the invention is a process for making a pharmaceutical composition comprising mixing a product prepared according to any of the processes described herein and a pharmaceutically acceptable carrier.

Exemplifying the invention are methods of treating a metabolic related disorder (selected from the group consisting of hyperlipidemia, type 1 diabetes, type 2 diabetes mellitus, idiopathic type 1 diabetes (Type 1 b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance) comprising administering to a subject in need thereof a therapeutically effective amount of any of the compounds or pharmaceutical compositions described above.

Another example of the present invention is the use of any of the compounds described herein in the preparation of a medicament for treating metabolic related disorders, in a subject in need thereof.

Another example of the present invention is the use of any of the compounds described herein in the preparation of a medicament for treating (a) type I diabetes, (b) type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, (j) syndrome X or (k), in a subject in need thereof.

### DETAILED DESCRIPTION OF THE INVENTION

The present invention is directed to processes for the preparation of compounds of formula (I)

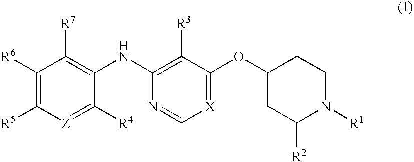

wherein R¹, R², R³, R⁴, R⁵, R⁶, R⁷, X and Z are as herein defined; or pharmaceutically acceptable salts, solvates or hydrates thereof. The compounds of formula (I) (for example, the compound of formula (I-S)) are glucose dependent insulintropic receptor agonists useful in the treatment of metabolic related disorders.

The processes of the present invention do not require the use of palladium catalysts which are both expensive and difficult to remove from isolated product. The processes of present invention are therefore advantageous for commercial or large scale manufacture of the compounds of formula (I), and more particularly the compound of formula (I-S).

The present invention is further directed to a compound prepared according to any of the processes described herein. The present invention is further directed to methods for the treatment of metabolic related disorders comprising administering to a subject in need thereof, a therapeutically effective amount of a compound prepared according to any of the processes described herein.

In an embodiment, the present invention is directed to the use of a compound prepared according to any of the processes described herein for the treatment of a metabolic-related disorder selected from the group consisting of type I diabetes, type II diabetes, inadequate glucose tolerance, insulin resistance, hyperglycemia, hyperlipidemia, hypertriglyceridemia, hypercholesterolemia, dyslipidemia and syndrome X; preferably, type II diabetes.

In another embodiment, the present invention is directed to the use of a compound prepared according to any of the processes described herein for the treatment of a metabolic-related disorder, wherein the metabolic related disorder is obesity.

In another embodiment, the present invention is directed to the use of a compound prepared according to any of the processes described herein for reducing food intake, inducing satiety, controlling weight gain, reducing weight gain

Some embodiments of the present invention pertain to synthesis of compounds wherein X is N. Some embodiments of the present invention pertain to synthesis of synthesis of compounds wherein X is CR₈. In some embodiments, R₈ is H or F.

Some embodiments of the present invention pertain to synthesis of compounds wherein Z is CH. Some embodiments of the present invention pertain to synthesis of compounds wherein Z is N.

Some embodiments of the present invention pertain to synthesis of compounds wherein R¹ is carbo-C₁₋₆-alkoxy optionally substituted with C₃₋₅cycloalkyl.

Some embodiments of the present invention pertain to synthesis of compounds wherein R¹ is selected form the group consisting of C(O)OCH₂CH₃, C(O)OCH(CH₃)₂, C(O)OCH(CH₃)(CH₂CH₃), C(O)OCH₂-cyclopropyl, C(O)OCH(CH₃)(cyclopropyl), and C(O)OCH(CH₂CH₃)₂. Some embodiments of the present invention pertain to synthesis of compounds wherein R¹ is selected form the group consisting of C(O)OCH₂CH₃, C(O)OCH(CH₃)₂, C(O)OCH(CH₃)(CH₂CH₃), C(O)OCH₂-cyclopropyl and C(O)OCH(CH₃)(cyclopropyl); these can be represented by the respective formulae:

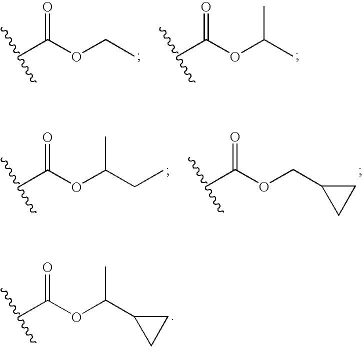

Some embodiments of the present invention pertain to synthesis of compounds wherein R¹ is oxadiazolyl optionally substituted with one C₁₋₄ alkyl group. Some embodiments of the present invention pertain to synthesis of compounds wherein R¹ is 5-isopropyl-[1,2,4]oxadiazol-3-yl. Some embodiments of the present invention pertain to synthesis of compounds wherein R¹ is pyrimidinyl optionally substituted with one C₁₋₄ alkoxy group. Some embodiments of the present invention pertain to synthesis of compounds wherein R¹ is 5-methoxy-pyrimidin-2-yl.

Some embodiments of the present invention pertain to synthesis of compounds wherein R² is H. Some embodiments of the present invention pertain to synthesis of compounds wherein R² is CH₃.

Some embodiments of the present invention pertain to synthesis of compounds wherein R³ is C₁₋₄alkoxy. Some embodiments of the present invention pertain to synthesis of compounds wherein R³ is OCH₃ or OCH₂CH₃.

Some embodiments of the present invention pertain to synthesis of compounds wherein R³ is OCH₃. Some embodiments of the present invention pertain to synthesis of compounds wherein R³ is OH or O—C≡CH.

Some embodiments of the present invention pertain to synthesis of compounds wherein R⁴ is selected from the group consisting of H, OCH₃, CH₃, CH₂CH₃, F, Cl and C≡CH. Some embodiments of the present invention pertain to synthesis of compounds wherein R⁴ is CH₃.

Some embodiments of the present invention pertain to synthesis of compounds wherein R⁵ is selected from the group consisting of OCH₂CH₂CH₃,

OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂, S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, CH₂C(O)N(CH₃)₂, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂, S(O)₂NH₂, CH₃, SCH₂CH₂CH₃, S(O)₂CH₂CH₂CH₃, SCH₂CH₃, SCH(CH₃)₂, S(O)₂CH(CH₃)₂, and CH₂OH.

Some embodiments of the present invention pertain to synthesis of compounds wherein R⁵ is selected from the group consisting of OCH₂CH₂CH₃, OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂,

S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, CH₂C(O)N(CH₃)₂, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂ and S(O)₂NH₂.

Some embodiments of the present invention pertain to synthesis of compounds wherein R⁵ is selected from the group consisting of OCH₂CH₂CH₂OH, S(O)₂CH₃, CH₂CH₂S(O)₂CH₃, NHCH₂CH₂OH, cyano, CH₂CH₂OH, CH₂CH₂CH(CH₃)OH, CH₂CH₂OP(O)(OH)₂, S(O)₂NHC(O)CH₂CH₃, CH₂CH₂CH₂OH, S(O)₂CH₂CH₃, NHCH₂CH(OH)CH₂OH, amino, NHCH₂CH₃, NHCH(CH₃)₂ and NHCH(CH₃)CH₂CH₃. Some embodiments of the present invention pertain to synthesis of compounds wherein R⁵ is a group other than —CH₂—R¹⁰, wherein R¹⁰ is selected from the group consisting of C₁₋₄alkylcarboxamide, C₁₋₄alkylsulfonyl, di-C₁₋₄-alkylcarboxamide, and phosphonooxy. In some embodiments, R⁵ is a group other than —CH₂—R¹⁰, wherein R¹⁰ is C₁₋₄alkylcarboxamide. In some embodiments, R⁵ is a group other than —CH₂—R¹⁰, wherein R¹⁰ is C₁₋₄alkylsulfonyl. In some embodiments, R⁵ is a group other than —CH₂—R₁₀, wherein R¹⁰ is di-C₁₋₄-alkylcarboxamide. In some embodiments, R⁵ is a group other than —CH₂—R¹⁰, wherein R¹⁰ is phosphonooxy.

Some embodiments of the present invention pertain to synthesis of compounds wherein R⁵ is selected from the group consisting of OCH₂CH₂CH₃, OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂, S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂ and S(O)₂NH₂.

In some embodiment, the present invention is directed to processes for the preparation of compounds of formula (I) wherein R⁵ is other than a group of Formula (A). In some embodiments, the present invention is directed to processes for the preparation of compounds of formula (I) wherein R⁵ is a group of Formula (A):

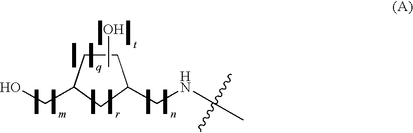

wherein “m”, “n” and “q” are each independently 0, 1, 2 or 3; “r” is 0, 1 or 2; and “t” is 0 or 1. In some embodiments, “m” and “n” are each independently 0 or 1. In some embodiments, “q” is 0 or 1 and “r” is 1 or 2. In some embodiments, “t” is 1. In some embodiments, “t” is 0.

Some embodiments, the present invention pertains to synthesis of compounds wherein R⁵ is a group of Formula (B):

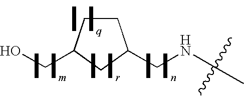

wherein “m”, “n”, “q” and “r” are as described herein, supra and infra.

Some embodiments of the present invention pertain to synthesis of compounds wherein R⁵ is selected from the group consisting of:

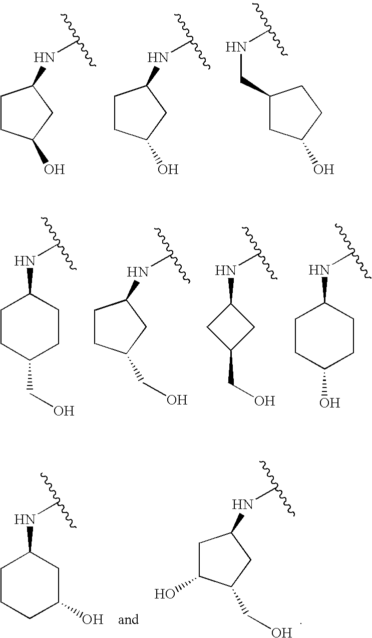

Some embodiments of the present invention pertain to synthesis of compounds wherein R⁶ is H. Some embodiments of the present invention pertain to synthesis of compounds wherein R⁶ is F.

Some embodiments of the present invention pertain to synthesis of compounds wherein R⁷ is H. Some embodiments of the present invention pertain to synthesis of compounds wherein R⁷ is CH₃.

Some embodiments of the present invention pertain to synthesis of compounds having Formula (IIa):

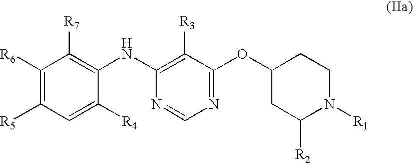

or a pharmaceutically acceptable salt, solvate or hydrate thereof;

wherein:

R¹ is carbo-C₁₋₆-alkoxy optionally substituted with C₃₋₅ cycloalkyl;

R² is H or CH₃;

R³ is C₁₋₄ alkoxy;

R⁴ is selected from the group consisting of H, OCH₃, CH₃, CH₂CH₃, F and Cl;

R⁵ is selected from the group consisting of OCH₂CH₂CH₃, OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂, S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, CH₂C(O)N(CH₃)₂, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂ and S(O)₂NH₂;

R₆ is H or F; and

R₇ is H or CH₃.

Some embodiments of the present invention pertain to synthesis of compounds having Formula (IIa):

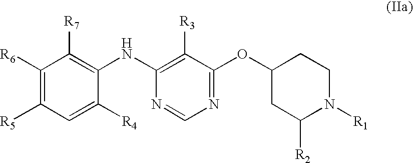

or a pharmaceutically acceptable salt, solvate or hydrate thereof;

wherein:

R¹ is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁵ is selected from the group consisting of OCH₂CH₂CH₂OH, S(O)₂CH₃, CH₂CH₂S(O)₂CH₃, NHCH₂CH₂OH, cyano, CH₂CH₂OH, CH₂CH₂CH(CH₃)OH, CH₂CH₂OP(O)(OH)₂, S(O)₂NHC(O)CH₂CH₃, CH₂CH₂CH₂OH, S(O)₂CH₂CH₃, NHCH₂CH(OH)CH₂OH and S(O)₂NH₂;

R⁶ is H or F; and

R⁷ is H or CH₃.

Some embodiments of the present invention pertain to synthesis of compounds having Formula (IIc):

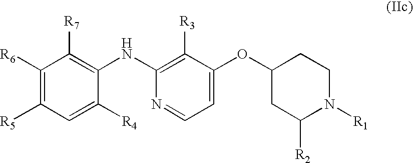

or a pharmaceutically acceptable salt, solvate or hydrate thereof;

wherein:

R¹ is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁵ is selected from the group consisting of OCH₂CH₂CH₃, OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂, S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, CH₂C(O)N(CH₃)₂, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂ and S(O)₂NH₂;

R⁶ is H or F; and

R⁷ is H or CH₃.

Some embodiments of the present invention pertain to synthesis of compounds having Formula (IIc):

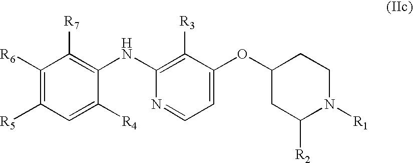

or a pharmaceutically acceptable salt, solvate or hydrate thereof;

wherein:

R¹ is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁵ is selected from the group consisting of OCH₂CH₂CH₂OH, S(O)₂CH₃, CH₂CH₂S(O)₂CH₃, NHCH₂CH₂OH, cyano, CH₂CH₂OH, CH₂CH₂CH(CH₃)OH, CH₂CH₂OP(O)(OH)₂, S(O)₂NHC(O)CH₂CH₃, CH₂CH₂CH₂OH, S(O)₂CH₂CH₃, NHCH₂CH(OH)CH₂OH and S(O)₂NH₂;

R⁶ is H or F; and

R⁷ is H or CH₃.

Some embodiments of the present invention pertain to synthesis of compounds having Formula (IIe):

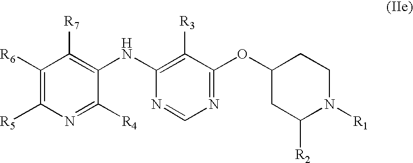

or a pharmaceutically acceptable salt, solvate or hydrate thereof;

wherein:

R^{al} is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁵ is selected from the group consisting of OCH₂CH₂CH₃, OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂, S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, CH₂C(O)N(CH₃)₂, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂ and S(O)₂NH₂;

R⁶ is H or F; and

R⁷ is H or CH₃.

Some embodiments of the present invention pertain to synthesis of compounds having Formula (IIe):

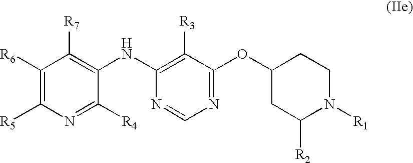

or a pharmaceutically acceptable salt, solvate or hydrate thereof;

wherein:

R¹ is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁵ is selected from the group consisting of OCH₂CH₂CH₂OH, S(O)₂CH₃, CH₂CH₂S(O)₂CH₃, NHCH₂CH₂OH, cyano, CH₂CH₂OH, CH₂CH₂CH(CH₃)OH, CH₂CH₂OP(O)(OH)₂, S(O)₂NHC(O)CH₂CH₃, CH₂CH₂CH₂OH, S(O)₂CH₂CH₃, NHCH₂CH(OH)CH₂OH and S(O)₂NH₂;

R⁶ is H or F; and

R⁷ is H or CH₃.

Some embodiments of the present invention pertain to synthesis of compounds having Formula (IIg):

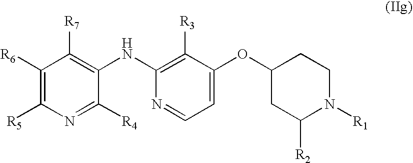

or a pharmaceutically acceptable salt, solvate or hydrate thereof;

wherein:

R¹ is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁵ is selected from the group consisting of OCH₂CH₂CH₃, OCH₂CH₂CH₂OH, S(O)₂CH₃, CH₂CH₂S(O)₂CH₃, NHCH₂CH₂OH, cyano, CH₂CH₂OCH₃, CH₂CH₂OH, CH₂CH₂CH(CH₃)OH, CH₂CH₂OP(O)(OH)₂, S(O)₂NHC(O)CH₂CH₃, CH₂CH₂O-cyclopropyl, NHCH₂CH₂OCH₃, OCH₂CH₂S(O)₂CH₃, NHCH₂CH(CH₃)OH, CH₂CH₂CH₂OH, CH₂CH₂CH₂OP(O)(OH)₂, NHCH₂CH(CH₃)S(O)₂CH₃, N(CH₃)CH₂CH(CH₃)S(O)₂CH₃, 3-methanesulfonyl-pyrrolidin-1-yl, 3-methanesulfonyl-piperidin-1-yl, CH₂C(O)N(CH₃)₂, 3-methanesulfonyl-azetidin-1-yl, CH₂C(O)NHCH₂CH₂OH, SCH₂CH₂OH, S(O)₂CH₂CH₂OP(O)(OH)₂, S(O)₂CH₂CH₃, NHCH₂CH(OH)CH₂OH, S(O)₂CH₂CH₂OH, OCH₂CH₂OP(O)(OH)₂, OCH₂CH₂CH₂OP(O)(OH)₂ and S(O)₂NH₂;

R⁶ is H or F; and

R⁷ is H or CH₃.

Some embodiments of the present invention pertain to synthesis of compounds having Formula (IIg):

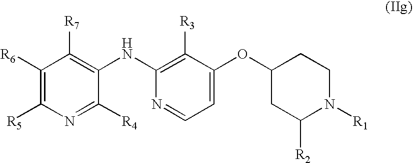

or a pharmaceutically acceptable salt, solvate or hydrate thereof;

wherein:

R¹ is carbo-C₁₋₆-alkoxy optionally substituted with C₃₋₅ cycloalkyl;

R² is H or CH₃;

R³ is C₁₋₄ alkoxy;

R⁴ is selected from the group consisting of H, OCH₃, CH₃, CH₂CH₃, F and Cl;

R⁵ is selected from the group consisting of OCH₂CH₂CH₂OH, S(O)₂CH₃, CH₂CH₂S(O)₂CH₃, NHCH₂CH₂OH, cyano, CH₂CH₂OH, CH₂CH₂CH(CH₃)OH, CH₂CH₂OP(O)(OH)₂, S(O)₂NHC(O)CH₂CH₃, CH₂CH₂CH₂OH, S(O)₂CH₂CH₃, NHCH₂CH(OH)CH₂OH and S(O)₂NH₂;

R⁶ is H or F; and

R⁷ is H or CH₃.

Some embodiments of the present invention pertain to synthesis of synthesis of compounds having Formula (IIi):

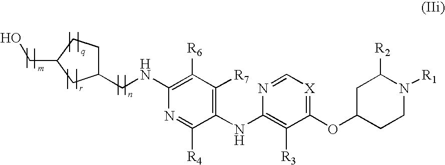

or a pharmaceutically acceptable salt, solvate or hydrate thereof;

wherein:

“m” and “n” are each independently 0 or 1;

“q” is 0 or 1;

“r” is 1 or 2;

X is N or O;

R¹ is carbo-C₁₋₆-alkoxy optionally substituted with C₃₋₅cycloalkyl;

R² is H or CH₃;

R³ is C₁₋₄alkoxy;

R⁴ is selected from the group consisting of H, OCH₃, CH₃, CH₂CH₃, F and Cl;

R⁶ is H or F; and

R⁷ is H or CH₃.

Some embodiments of the present invention pertain to the synthesis of every combination of one or more compounds selected from the following group in Table 1.

TABLE 1
Representative Compounds of Formula (I)

| Cmpd No. | Structure | Chemical Name |
|---|---|---|
| 1 | 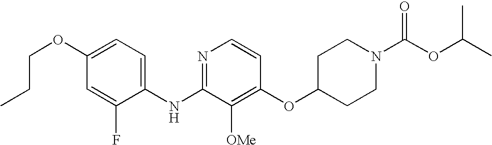 | 4-[2-(2-Fluoro-4-propoxy- phenylamino)-3-methoxy- pyridin-4-yloxy]-piperidine-1- carboxylic acid isopropyl ester |
| 2 | 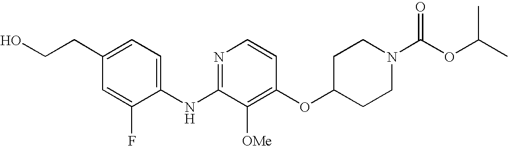 | 4-{2-[2-Fluoro-4-(2-hydroxy- ethyl)-phenylamino]-3-methoxy- pyridin-4-yloxy}-piperidine-1- carboxylic acid isopropyl ester |
| 3 | 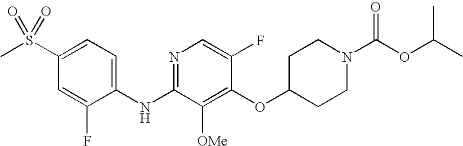 | 4-[5-Fluoro-2-(2-fluoro-4- methanesulfonyl-phenylamino)- 3-methoxy-pyridin-4-yloxy]- piperidine-1-carboxylic acid isopropyl ester |
| 4 | 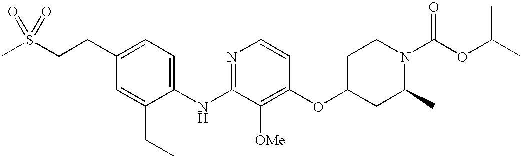 | (S)-4-{2-[2-Ethyl-4-(2- methanesulfonyl-ethyl)- phenylamino]-3-methoxy-pyridin- 4-yloxy}-2-methyl-piperidine-1- carboxylic acid isopropyl ester |
| 5 | 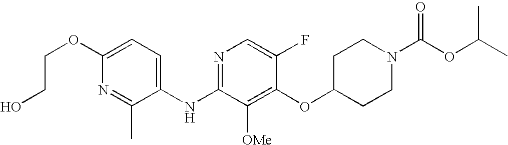 | 4-{5-Fluoro-2-[6-(2-hydroxy- ethoxy)-2-methyl-pyridin-3- ylamino]-3-methoxy-pyridin-4- yloxy}-piperidine-1-carboxylic acid isopropyl ester |
| 6 | 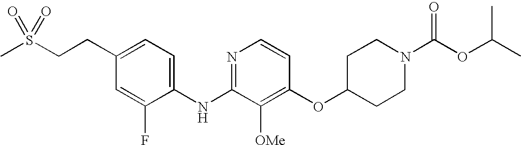 | 4-{2-[2-Fluoro-4-(2- methanesulfonyl-ethyl)- phenylamino]-3-methoxy-pyridin-4- yloxy}-piperidine-1-carboxylic acid isopropyl ester |
| 7 | 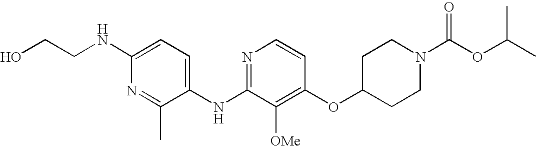 | 4-{2-[6-(2-Hydroxy-ethylamino)-2- methyl-pyridin-3-ylamino]-3- methoxy-pyridin-4-yloxy}-piperidine- 1-carboxylic acid isopropyl ester |
| 8 | 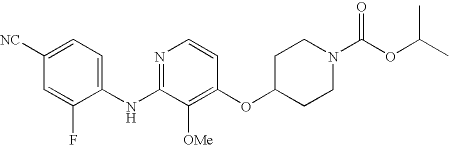 | 4-[2-(4-Cyano-2-fluoro- phenylamino)-3-methoxy-pyridin-4- yloxy]-piperidine-1-carboxylic acid isopropyl ester |
| 9 | 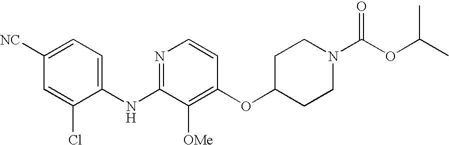 | 4-[2-(2-Chloro-4-cyano- phenylamino)-3-methoxy-pyridin-4- yloxy]-piperidine-1-carboxylic acid isopropyl ester |
| 10 | 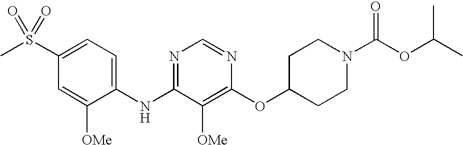 | 4-[6-(4-Methanesulfonyl-2-methoxy- phenylamino)-5-methoxy-pyrimidin- 4-yloxy]-piperidine-1-carboxylic acid isopropyl ester |
| 11 | 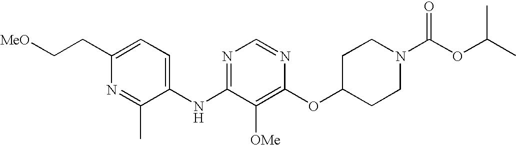 | 4-{5-Methoxy-6-[6-(2-methoxy- ethyl)-2-methyl-pyridin-3-ylamino]- pyrimidin-4-yloxy}-piperidine-1- carboxylic acid isopropyl ester |
| 12 | 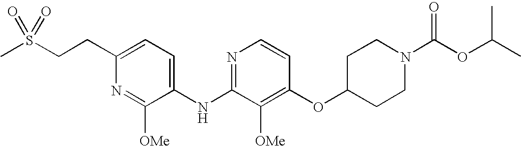 | 4-{2-[6-(2-Methanesulfonyl-ethyl)-2- methoxy-pyridin-3-ylamino]-3- methoxy-pyridin-4-yloxy}-piperidine- 1-carboxylic acid isopropyl ester |
| 13 | 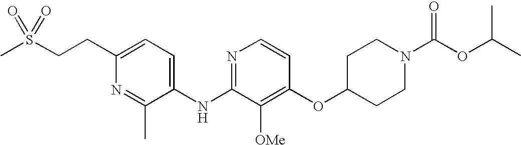 | 4-{2-[6-(2-Methanesulfonyl-ethyl)-2- methyl-pyridin-3-ylamino]-3- methoxy-pyridin-4-yloxy}-piperidine- 1-carboxylic acid isopropyl ester |
| 14 | 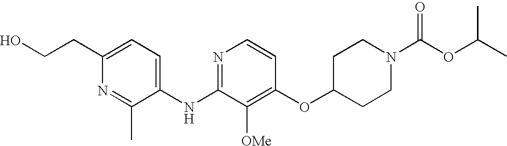 | 4-{2-[6-(2-Hydroxy-ethyl)-2-methyl- pyridin-3-ylamino]-3-methoxy- pyridin-4-yloxy}-piperidine-1- carboxylic acid isopropyl ester |
| 15 | 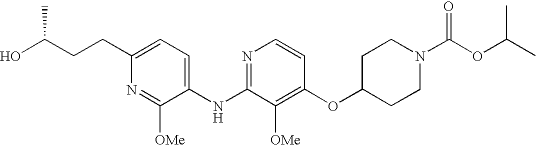 | (R)-4-{2-[6-(3-Hydroxy-butyl)-2- methoxy-pyridin-3-ylamino]-3- methoxy-pyridin-4-yloxy}-piperidine- 1-carboxylic acid isopropyl ester |
| 16 | 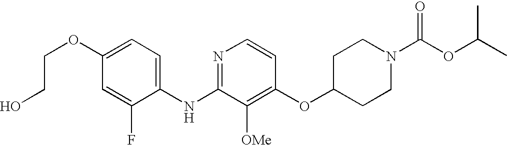 | 4-{2-[2-Fluoro-4-(2-hydroxy- ethoxy)-phenylamino]-3-methoxy- pyridin-4-yloxy}-piperidine-1- carboxylic acid isopropyl ester |
| 17 | 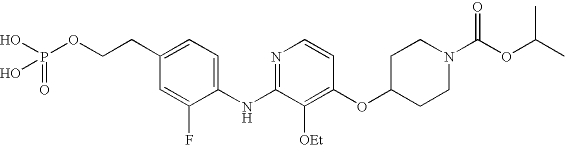 | 4-{3-Ethoxy-2-[2-fluoro-4-(2- phosphonooxy-ethyl)-phenylamino]- pyridin-4-yloxy}-piperidine-1- carboxylic acid isopropyl ester |
| 18 | 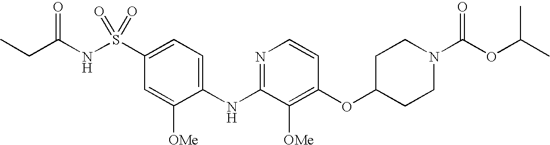 | 4-[3-Methoxy-2-(2-methoxy-4- propionylsulfamoyl-phenylamino)- pyridin-4-yloxy]-piperidine-1- carboxylic acid isopropyl ester |
| 19 | 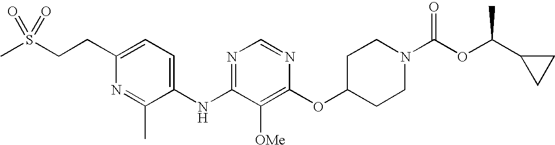 | (S)-4-{6-[6-(2-Methanesulfonyl- ethyl)-2-methyl-pyridin-3-ylamino]- 5-methoxy-pyrimidin-4-yloxy}- piperidine-1-carboxylic acid 1- cyclopropyl-ethyl ester |
| 20 | 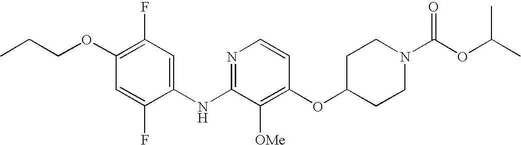 | 4-[2-(2,5-Difluoro-4-propoxy- phenylamino)-3-methoxy-pyridin-4- yloxy]-piperidine-1-carboxylic acid isopropyl ester |
| 21 | 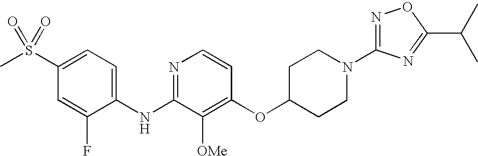 | (2-Fluoro-4-methanesulfonyl- phenyl)-{4-[1-(5-isopropyl- [1,2,4]oxadiazol-3-yl)-piperidin-4- yloxy]-3-methoxy-pyridin-2-yl}- amine |
| 22 | 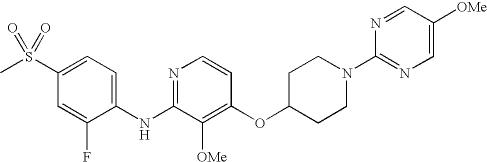 | (2-Fluoro-4-methanesulfonyl- phenyl)-{3-methoxy-4-[1-(5- methoxy-pyrimidin-2-yl)-piperidin-4- yloxy]-pyridin-2-yl}-amine |
| 23 | 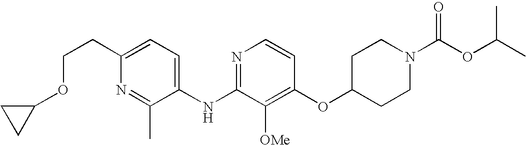 | 4-{2-[6-(2-Cyclopropoxy-ethyl)-2- methyl-pyridin-3-ylamino]-3- methoxy-pyridin-4-yloxy}-piperidine- 1-carboxylic acid isopropyl ester |
| 24 | 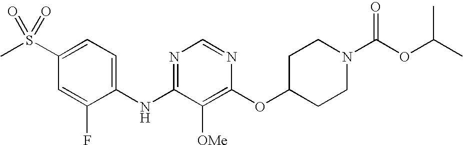 | 4-[6-(2-Fluoro-4-methanesulfonyl- phenylamino)-5-methoxy-pyrimidin- 4-yloxy]-piperidine-1-carboxylic acid isopropyl ester |
| 25 | 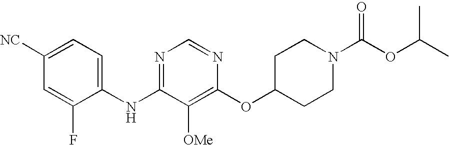 | 4-[6-(4-Cyano-2-fluoro- phenylamino)-5-methoxy-pyrimidin- 4-yloxy]-piperidine-1-carboxylic acid isopropyl ester |
| 26 | 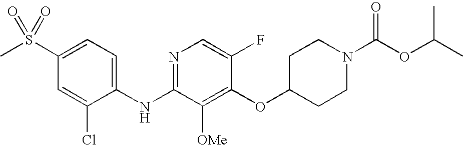 | 4-[2-(2-Chloro-4-methanesulfonyl- phenylamino)-5-fluoro-3-methoxy- pyridin-4-yloxy]-piperidine-1- carboxylic acid isopropyl ester |
| 27 | 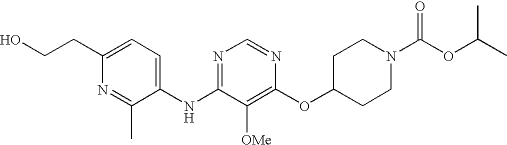 | 4-{6-[6-(2-Hydroxy-ethyl)-2-methyl- pyridin-3-ylamino]-5-methoxy- pyrimidin-4-yloxy}-piperidine-1- carboxylic acid isopropyl ester |
| 28 | 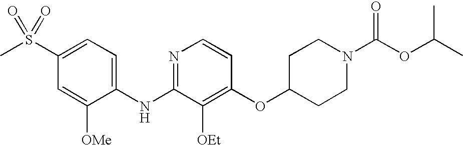 | 4-[3-Ethoxy-2-(4-methanesulfonyl- 2-methoxy-phenylamino)-pyridin-4- yloxy]-piperidine-1-carboxylic acid isopropyl ester |
| 29 | 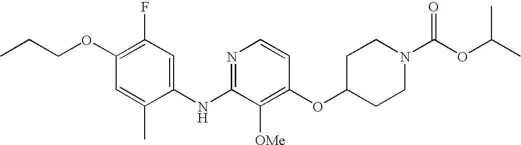 | 4-[2-(5-Fluoro-2-methyl-4-propoxy- phenylamino)-3-methoxy-pyridin-4- yloxy]-piperidine-1-carboxylic acid isopropyl ester |
| 30 | 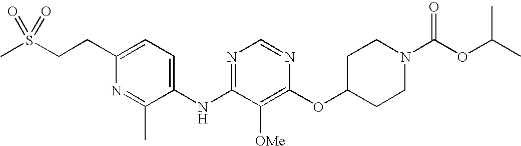 | 4-{6-[6-(2-Methanesulfonyl-ethyl)-2- methyl-pyridin-3-ylamino]-5- methoxy-pyrimidin-4-yloxy}- piperidine-1-carboxylic acid isopropyl ester |
| 31 | 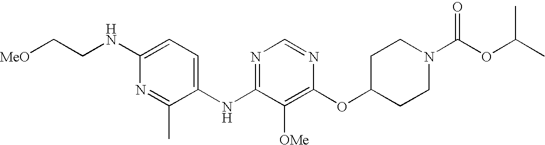 | 4-{5-Methoxy-6-[6-(2-methoxy- ethylamino)-2-methyl-pyridin-3- ylamino]-pyrimidin-4-yloxy}- piperidine-1-carboxylic acid isopropyl ester |
| 32 | 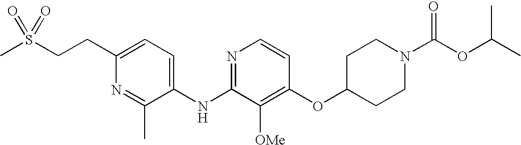 | 4-{2-[6-(2-Methanesulfonyl-ethyl)-2- methyl-pyridin-3-ylamino]-3- methoxy-pyridin-4-yloxy}-piperidine- 1-carboxylic acid isopropyl ester |
| 33 | 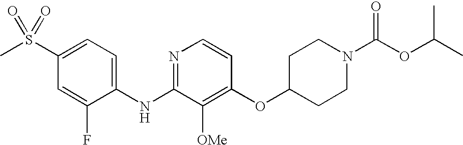 | 4-[2-(2-Fluoro-4-methanesulfonyl- phenylamino)-3-hydroxy-pyridin-4- yloxy]-piperidine-1-carboxylic acid isopropyl ester |
| 34 | 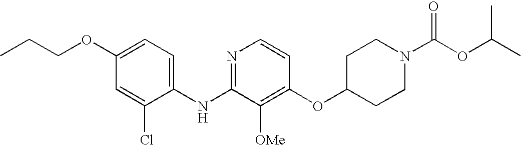 | 4-[2-(2-Chloro-4-propoxy- phenylamino)-3-methoxy-pyridin-4- yloxy]-piperidine-1-carboxylic acid isopropyl ester |
| 35 | 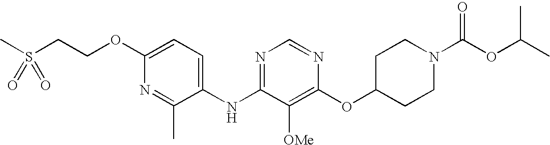 | 4-{6-[6-(2-Methanesulfonyl-ethoxy)- 2-methyl-pyridin-3-ylamino]-5- methoxy-pyrimidin-4-yloxy}- piperidine-1-carboxylic acid isopropyl ester |
| 36 | 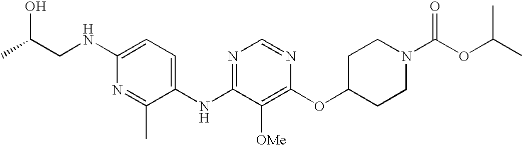 | (S)-4-{6-[6-(2-Hydroxy- propylamino)-2-methyl-pyridin-3- ylamino]-5-methoxy-pyrimidin-4- yloxy}-piperidine-1-carboxylic acid isopropyl ester |
| 37 | 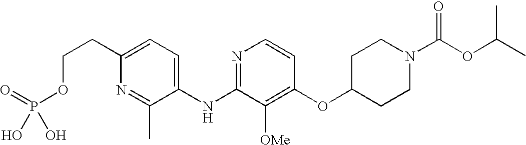 | 4-{3-Methoxy-2-[2-methyl-6-(2- phosphonooxy-ethyl)-pyridin-3- ylamino]-pyridin-4-yloxy}-piperidine- 1-carboxylic acid isopropyl ester |
| 38 | 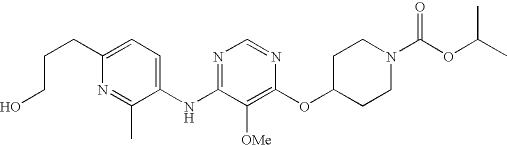 | 4-{6-[6-(3-Hydroxy-propyl)-2- methyl-pyridin-3-ylamino]-5- methoxy-pyrimidin-4-yloxy}- piperidine-1-carboxylic acid isopropyl ester |
| 39 | 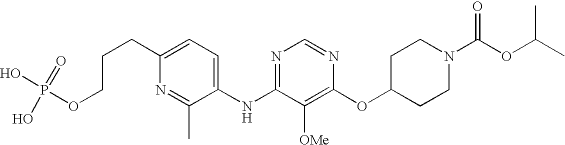 | 4-{5-Methoxy-6-[2-methyl-6-(3- phosphonooxy-propyl)-pyridin-3- ylamino]-pyrimidin-4-yloxy}- piperidine-1-carboxylic acid isopropyl ester |
| 40 | 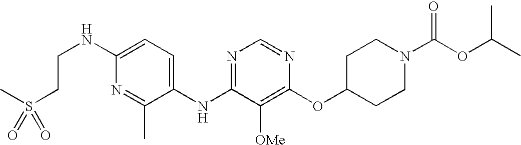 | 4-{6-[6-(2-Methanesulfonyl- ethylamino)-2-methoxy-pyridin-3- ylamino]-5-methoxy-pyrimidin-4- yloxy}-piperidine-1-carboxylic acid isopropyl ester |
| 41 | 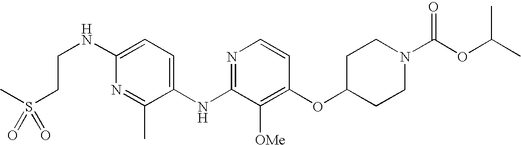 | 4-{2-[6-(2-Methanesulfonyl- ethylamino)-2-methyl-pyridin-3- ylamino]-3-methoxy-pyridin-4- yloxy}-piperidine-1-carboxylic acid isopropyl ester |
| 42 | 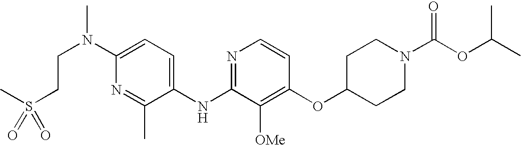 | 4-(2-{6-[(2-Methanesulfonyl-ethyl)- methyl-amino]-2-methyl-pyridin-3- ylamino}-3-methoxy-pyridin-4- yloxy)-piperidine-1-carboxylic acid isopropyl ester |
| 43 | 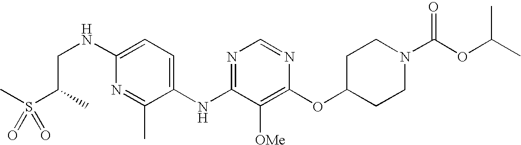 | (S)-4-{6-[6-(2-Methanesulfonyl- propylamino)-2-methyl-pyridin-3- ylamino]-5-methoxy-pyrimidin-4- yloxy}-piperidine-1-carboxylic acid isopropyl ester |
| 44 | 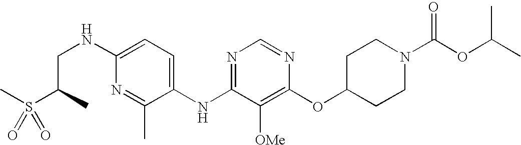 | (R)-4-{6-[6-(2-Methanesulfonyl- propylamino)-2-methyl-pyridin-3- ylamino]-5-methoxy-pyrimidin-4- yloxy}-piperidine-1-carboxylic acid isopropyl ester |
| 45 | 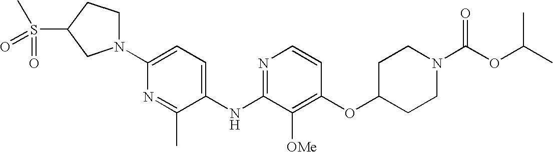 | 4-{2-[6-(3-Methanesulfonyl- pyrrolidin-1-yl)-2-methyl-pyridin-3- ylamino]-3-methoxy-pyridin-4- yloxy}-piperidine-1-carboxylic acid isopropyl ester |
| 46 | 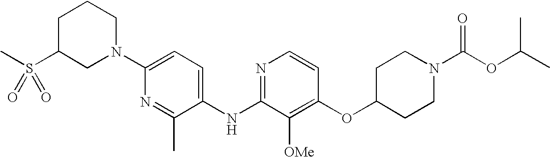 | 4-[2-(3-Methanesulfonyl-6′-methyl- 3,4,5,6-tetrahydro-2H- [1,2′]bipyridinyl-5′-ylamino)-3- methoxy-pyridin-4-yloxy]-piperidine- 1-carboxylic acid isopropyl ester |
| 47 | 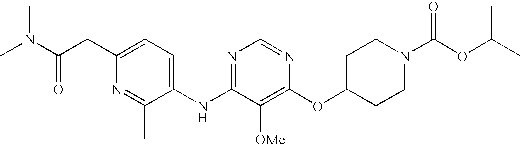 | 4-[6-(6-Dimethylcarbamoylmethyl- 2-methyl-pyridin-3-ylamino)-5- methoxy-pyrimidin-4-yloxy]- piperidine-1-carboxylic acid isopropyl ester |
| 48 | 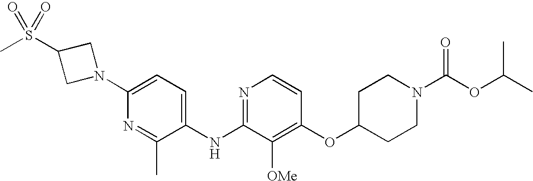 | 4-{2-[6-(3-Methanesulfonyl-azetidin- 1-yl)-2-methyl-pyridin-3-ylamino]-3- methoxy-pyridin-4-yloxy}-piperidine- 1-carboxylic acid isopropyl ester |
| 49 | 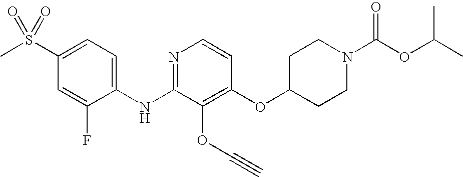 | 4-[3-Ethynyloxy-2-(2-fluoro-4- methanesulfonyl-phenylamino)- pyridin-4-yloxy]-piperidine-1- carboxylic acid isopropyl ester |
| 50 | 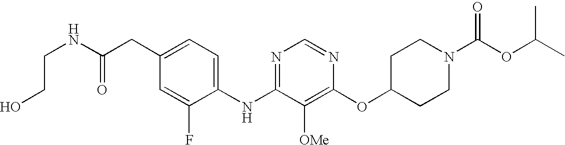 | 4-(6-{2-Fluoro-4-[(2-hydroxy- ethylcarbamoyl)-methyl]- phenylamino}-5-methoxy-pyrimidin- 4-yloxy)-piperidine-1-carboxylic acid isopropyl ester |
| 51 | 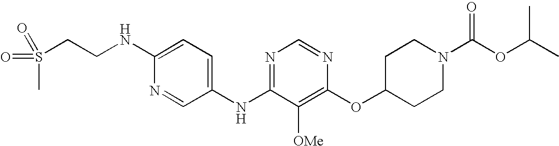 | 4-{6-[6-(2-Methanesulfonyl- ethylamino)-pyridin-3-ylamino]-5- methoxy-pyrimidin-4-yloxy}- piperidine-1-carboxylic acid isopropyl ester |
| 52 | 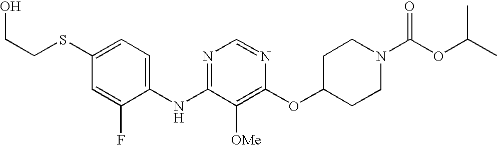 | 4-{6-[2-Fluoro-4-(2-hydroxy- ethylsulfanyl)-phenylamino]-5- methoxy-pyrimidin-4-yloxy}- piperidine-1-carboxylic acid isopropyl ester |
| 53 | 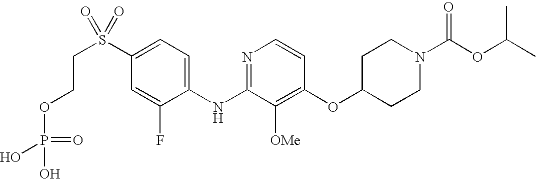 | 4-{2-[2-Fluoro-4-(2-phosphonooxy- ethanesulfonyl)-phenylamino]-3- methoxy-pyridin-4-yloxy}-piperidine- 1-carboxylic acid isopropyl ester |
| 54 | 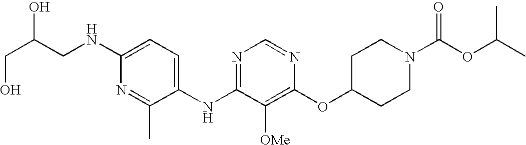 | 4-{6-[6-(2,3-Dihydroxy- propylamino)-2-methyl-pyridin-3- ylamino]-5-methoxy-pyrimidin-4- yloxy}-piperidine-1-carboxylic acid isopropyl ester |
| 55 | 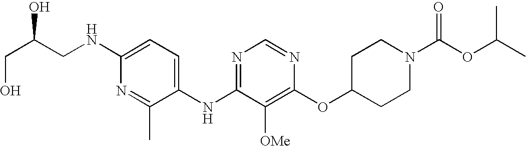 | (S)-4-{6-[6-(2 3-Dihydroxy- propylamino)-2-methyl-pyridin-3- ylamino]-5-methoxy-pyrimidin-4- yloxy}-piperidine-1-carboxylic acid isopropyl ester |
| 56 | 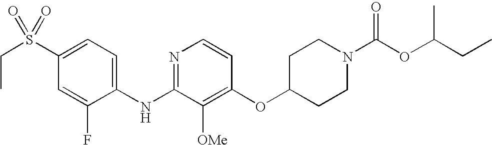 | 4-[2-(4-Ethanesulfonyl-2-fluoro- phenylamino)-3-methoxy-pyridin-4- yloxy]-piperidine-1-carboxylic acid sec-butyl ester |
| 57 | 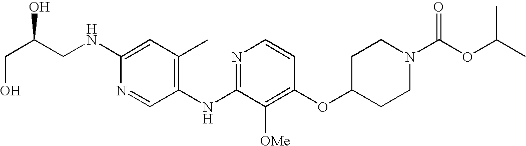 | 4-{2-[6-(2,3-Dihydroxy- propylamino)-4-methyl-pyridin-3- ylamino]-3-methoxy-pyridin-4- yloxy}-piperidine-1-carboxylic acid isopropyl ester |
| 58 | 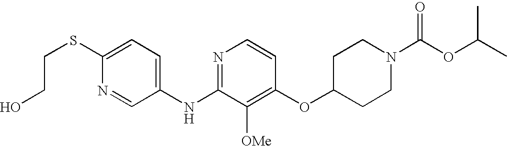 | 4-{2-[6-(2-Hydroxy-ethylsulfanyl)- pyridin-3-ylamino]-3-methoxy- pyridin-4-yloxy}-piperidine-1- carboxylic acid isopropyl ester |
| 59 | 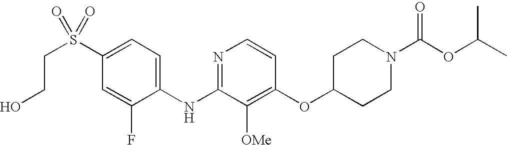 | 4-{2-[2-Fluoro-4-(2-hydroxy- ethanesulfonyl)-phenylamino]-3- methoxy-pyridin-4-yloxy}-piperidine- 1-carboxylic acid isopropyl ester |
| 60 | 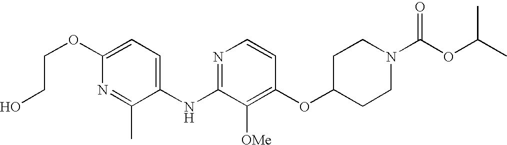 | 4-{2-[6-(2-Hydroxy-ethoxy)-2- methyl-pyridin-3-ylamino]-3- methoxy-pyridin-4-yloxy}-piperidine- 1-carboxylic acid isopropyl ester |
| 61 | 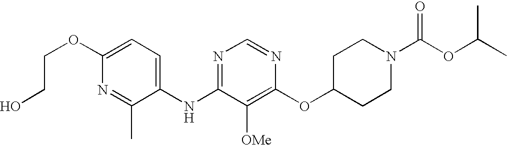 | 4-{6-[6-(2-Hydroxy-ethoxy)-2- methyl-pyridin-3-ylamino]-5- methoxy-pyrimidin-4-yloxy}- piperidine-1-carboxylic acid isopropyl ester |
| 62 | 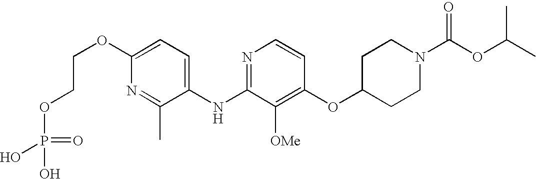 | 4-{3-Methoxy-2-[2-methyl-6-(2- phosphonooxy-ethoxy)-pyridin-3- ylamino]-pyridin-4-yloxy}-piperidine- 1-carboxylic acid isopropyl ester |
| 63 | 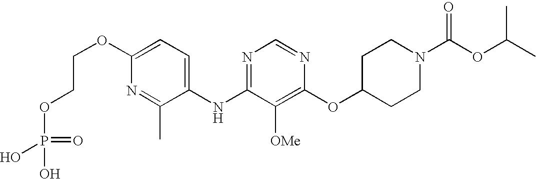 | 4-{5-Methoxy-6-[2-methyl-6-(2- phosphonooxy-ethoxy)-pyridin-3- ylamino]-pyrimidin-4-yloxy}- piperidine-1-carboxylic acid isopropyl ester |
| 64 | 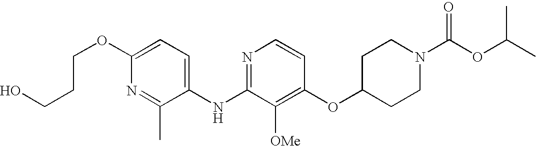 | 4-{2-[6-(3-Hydroxy-propoxy)-2- methyl-pyridin-3-ylamino]-3- methoxy-pyridin-4-yloxy}-piperidine- 1-carboxylic acid isopropyl ester |
| 65 | 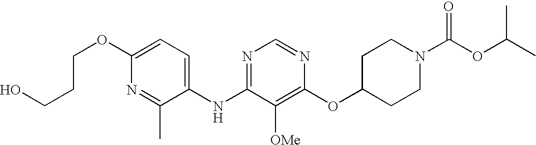 | 4-{6-[6-(3-Hydroxy-propoxy)-2- methyl-pyridin-3-ylamino]-5- methoxy-pyrimidin-4-yloxy}- piperidine-1-carboxylic acid isopropyl ester |
| 66 | 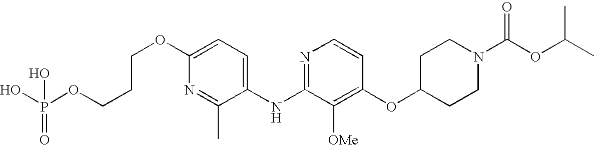 | 4-{3-Methoxy-2-[2-methyl-6-(3- phosphonooxy-propoxy)-pyridin-3- ylamino]-pyridin-4-yloxy}-piperidine- 1-carboxylic acid isopropyl ester |
| 67 | 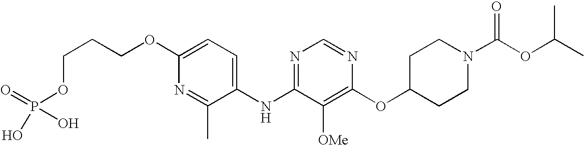 | 4-{5-Methoxy-6-[2-methyl-6-(3- phosphonooxy-propoxy)-pyridin-3- ylamino]-pyrimidin-4-yloxy}- piperidine-1-carboxylic acid isopropyl ester |
| 68 | 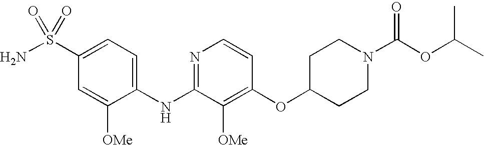 | 4-[3-Methoxy-2-(2-methoxy-4- sulfamoyl-phenylamino)-pyridin-4- yloxy]-piperidine-1-carboxylic acid isopropyl ester |
| 69 | 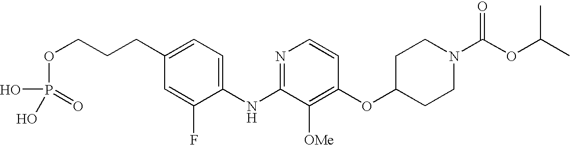 | 4-{2-[2-Fluoro-4-(3-phosphonooxy- propyl)-phenylamino]-3-methoxy- pyridin-4-yloxy}-piperidine-1- carboxylic acid isopropyl ester |
| 70 | 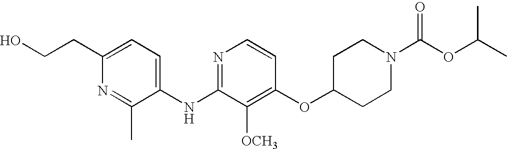 | 4-{2-[6-(2-Hydroxy-ethyl)-2-methyl- pyridin-3-ylamino]-3-methoxy- pyridin-4-yloxy}-piperidine-1- carboxylic acid isopropyl ester |
| 71 | 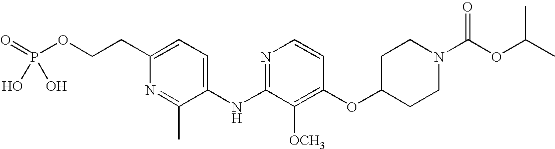 | 4-{3-Methoxy-2-[2-methyl-6-(2- phosphonooxy-ethyl)-pyridin-3- ylamino]-pyridin-4-yloxy}-piperidine- 1-carboxylic acid isopropyl ester |
| 72 | 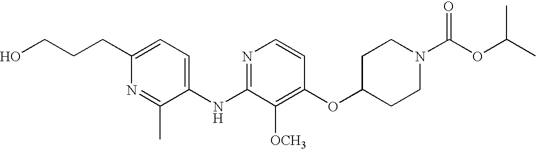 | 4-{2-[6-(3-Hydroxy-propyl)-2- methyl-pyridin-3-ylamino]-3- methoxy-pyridin-4-yloxy}-piperidine- 1-carboxylic acid isopropyl ester |
| 73 | 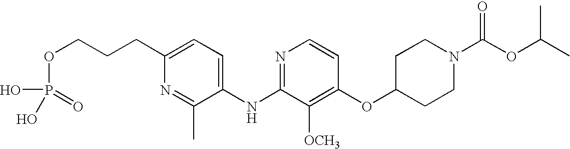 | 4-{3-Methoxy-2-[2-methyl-6-(3- phosphonooxy-propyl)-pyridin-3- ylamino]-pyridin-4-yloxy}-piperidine- 1-carboxylic acid isopropyl ester |
| 74 | 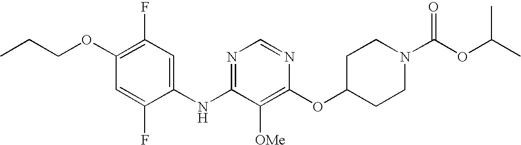 | 4-[6-(2,5-Difluoro-4-propoxy- phenylamino)-5-methoxy-pyrimidin- 4-yloxy]-piperidine-1-carboxylic acid isopropyl ester |
| 75 | 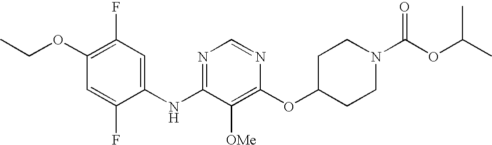 | 4-[6-(4-Ethoxy-2,5-difluoro- phenylamino)-5-methoxy-pyrimidin- 4-yloxy]-piperidine-1-carboxylic acid isopropyl ester |
| 77 | 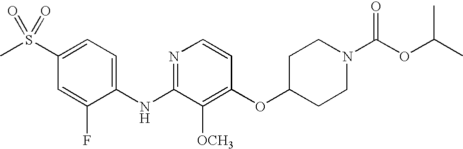 | 4-[2-(2-Fluoro-4-methanesulfonyl- phenylamino)-3-methoxy-pyridin-4- yloxy]-piperidine-1-carboxylic acid isopropyl ester |
| 78 | 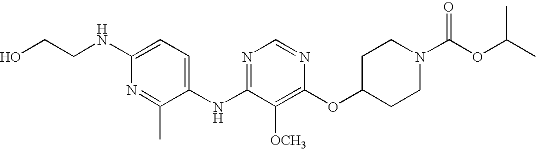 | 4-{6-[6-(2-Hydroxy-ethylamino)-2- methyl-pyridin-3-ylamino]-5- methoxy-pyrimidin-4-yloxy}- piperidine-1-carboxylic acid isopropyl ester |
| 79 | 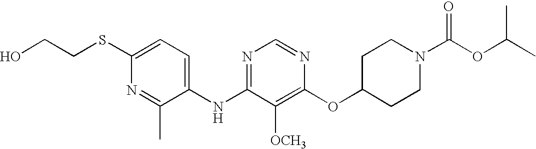 | 4-{6-[6-(2-Hydroxy-ethylsulfanyl)-2- methyl-pyridin-3-ylamino]-5- methoxy-pyrimidin-4-yloxy}- piperidine-1-carboxylic acid isopropyl ester |
| 80 | 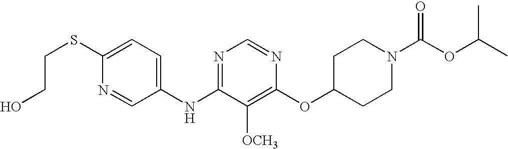 | 4-{6-[6-(2-Hydroxy-ethylsulfanyl)- pyridin-3-ylamino]-5-methoxy- pyrimidin-4-yloxy}-piperidine-1- carboxylic acid isopropyl ester |
| 81 | 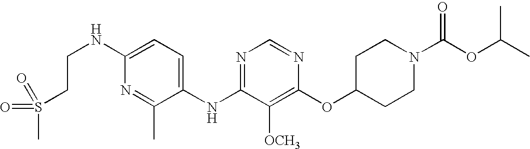 | 4-{6-[6-(2-Methanesulfonyl- ethylamino)-2-methyl-pyridin-3- ylamino]-5-methoxy-pyrimidin-4- yloxy}-piperidine-1-carboxylic acid isopropyl ester |
| 82 | 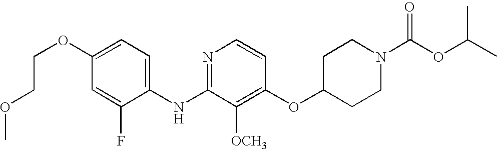 | 4-{2-[2-Fluoro-4-(2-methoxy- ethoxy)-phenylamino]-3-methoxy- pyridin-4-yloxy}-piperidine-1- carboxylic acid isopropyl ester |
| 83 | 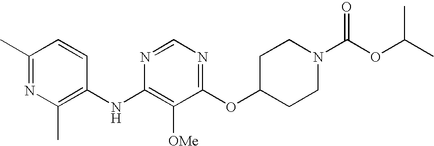 | 4-[6-(2,6-Dimethyl-pyridin-3- ylamino)-5-methoxy-pyrimidin-4- yloxy]-piperidine-1-carboxylic acid isopropyl ester |
| 84 | 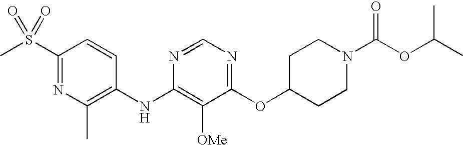 | 4-[6-(6-Methanesulfonyl-2-methyl- pyridin-3-ylamino)-5-methoxy- pyrimidin-4-yloxy]-piperidine-1- carboxylic acid isopropyl ester |
| 85 | 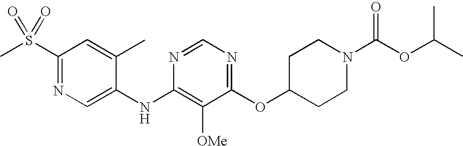 | 4-[6-(6-Methanesulfonyl-4-methyl- pyridin-3-ylamino)-5-methoxy- pyrimidin-4-yloxy]-piperidine-1- carboxylic acid isopropyl ester |
| 86 | 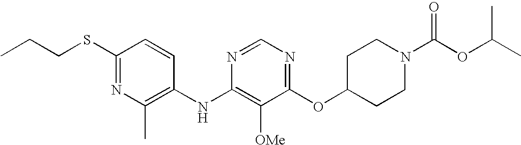 | 4-[5-Methoxy-6-(2-methyl-6- propylsulfanyl-pyridin-3-ylamino)- pyrimidin-4-yloxy]-piperidine-1- carboxylic acid isopropyl ester |
| 87 | 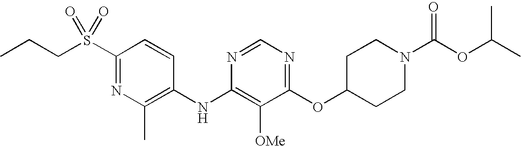 | 4-{5-Methoxy-6-[2-methyl-6- (propane-1-sulfonyl)-pyridin-3- ylamino]-pyrimidin-4-yloxy}- piperidine-1-carboxylic acid isopropyl ester |
| 88 | 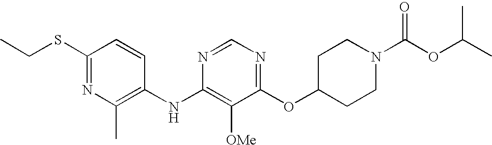 | 4-[6-(6-Ethylsulfanyl-2-methyl- pyridin-3-ylamino)-5-methoxy- pyrimidin-4-yloxy]-piperidine-1- carboxylic acid isopropyl ester |
| 89 | 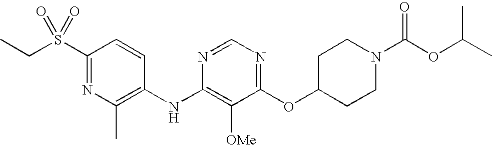 | 4-[6-(6-Ethanesulfonyl-2-methyl- pyridin-3-ylamino)-5-methoxy- pyrimidin-4-yloxy]-piperidine-1- carboxylic acid isopropyl ester |
| 90 | 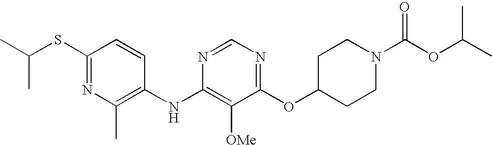 | 4-[6-(6-Isopropylsulfanyl-2-methyl- pyridin-3-ylamino)-5-methoxy- pyrimidin-4-yloxy]-piperidine-1- carboxylic acid isopropyl ester |
| 91 | 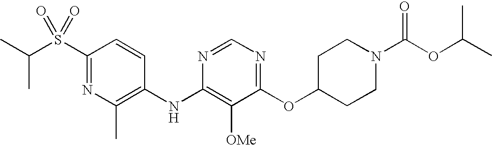 | 4-{5-Methoxy-6-[2-methyl-6- (propane-2-sulfonyl)-pyridin-3- ylamino]-pyrimidin-4-yloxy}- piperidine-1-carboxylic acid isopropyl ester |
| 92 | 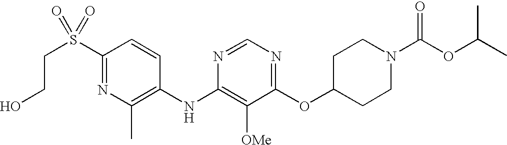 | 4-{6-[6-(2-Hydroxy-ethanesulfonyl)- 2-methyl-pyridin-3-ylamino]-5- methoxy-pyrimidin-4-yloxy}- piperidine-1-carboxylic acid isopropyl ester |
| 93 | 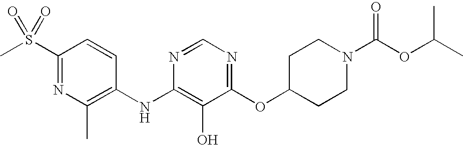 | 4-[5-Hydroxy-6-(6-methanesulfonyl- 2-methyl-pyridin-3-ylamino)- pyrimidin-4-yloxy]-piperidine-1- carboxylic acid isopropyl ester |
| 94 | 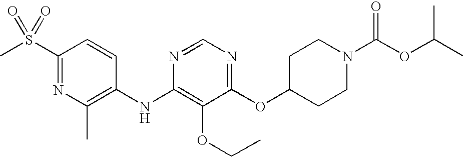 | 4-[5-Ethoxy-6-(6-methanesulfonyl- 2-methyl-pyridin-3-ylamino)- pyrimidin-4-yloxy]-piperidine-1- carboxylic acid isopropyl ester |
| 95 | 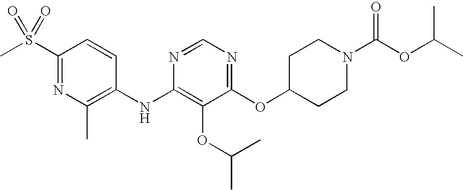 | 4-[5-Isopropoxy-6-(6- methanesulfonyl-2-methyl-pyridin- 3-ylamino)-pyrimidin-4-yloxy]- piperidine-1-carboxylic acid isopropyl ester |
| 96 | 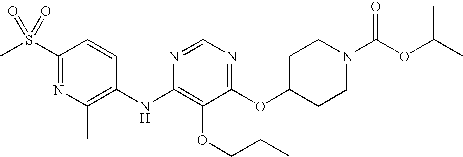 | 4-[6-(6-Methanesulfonyl-2-methyl- pyridin-3-ylamino)-5-propoxy- pyrimidin-4-yloxy]-piperidine-1- carboxylic acid isopropyl ester |
| 97 | 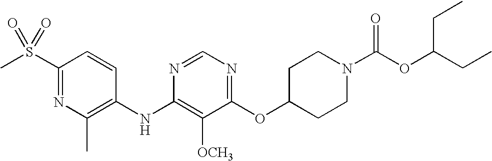 | 4-[6-(6-Methanesulfonyl-2-methyl- pyridin-3-ylamino)-5-methoxy- pyrimidin-4-yloxy]-piperidine-1- carboxylic acid 1-ethyl-propyl ester |
| 98 | 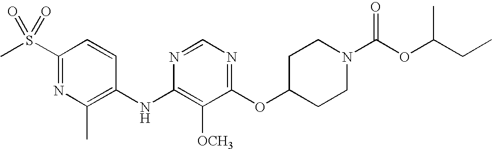 | 4-[6-(6-Methanesulfonyl-2-methyl- pyridin-3-ylamino)-5-methoxy- pyrimidin-4-yloxy]-piperidine-1- carboxylic acid sec-butyl ester |
| 99 | 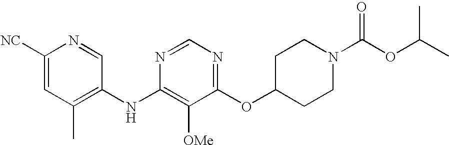 | 4-[6-(6-Cyano-4-methyl-pyridin-3- ylamino)-5-methoxy-pyrimidin-4- yloxy]-piperidine-1-carboxylic acid isopropyl ester |
| 100 | 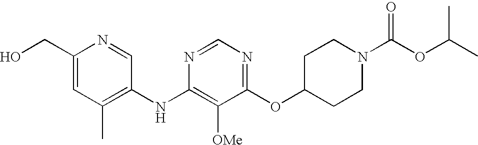 | 4-[6-(6-Hydroxymethyl-4-methyl- pyridin-3-ylamino)-5-methoxy- pyrimidin-4-yloxy]-piperidine-1- carboxylic acid isopropyl ester |
| 101 | 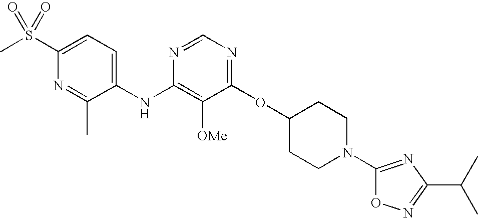 | {6-[1-(3-Isopropyl-[1,2,4]oxadiazol- 5-yl)-piperidin-4-yloxy]-5-methoxy- pyrimidin-4-yl}-(6-methanesulfonyl- 2-methyl-pyridin-3-yl)-amine |
| 102 | 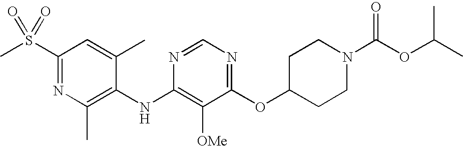 | 4-[6-(6-Methanesulfonyl-2,4- dimethyl-pyridin-3-ylamino)-5- methoxy-pyrimidin-4-yloxy]- piperidine-1-carboxylic acid isopropyl ester |
| 103 | 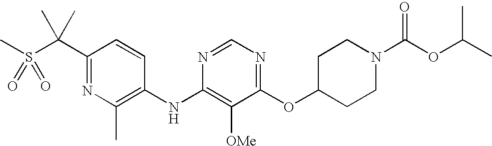 | 4-{6-[6-(1-Methanesulfonyl-1- methyl-ethyl)-2-methyl-pyridin-3- ylamino]-5-methoxy-pyrimidin-4- yloxy}-piperidine-1-carboxylic acid isopropyl ester |

The term “C₁₋₄acyl” refers to a C₁₋₆ alkyl radical attached directly to the carbon of a carbonyl group wherein the definition for alkyl is as described herein; some examples include, but not limited to, acetyl, propionyl, n-butanoyl, iso-butanoyl, sec-butanoyl, t-butanoyl (also referred to as pivaloyl) and the like.

The term “C₁₋₄acylsulfonamide” refers to a C₁₋₄acyl attached directly to the nitrogen of the sulfonamide, wherein the definitions for C₁₋₄acyl and sulfonamide have the same meaning as described herein, and a C₁₋₄acylsulfonamide group can be represented by the following formula:

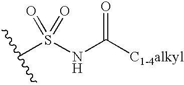

Some embodiments of the present invention are when acylsulfonamide is a C₁₋₃acylsulfonamide, some embodiments are C₁₋₂acylsulfonamide and some embodiments are C₁acylsulfonamide. Examples of an acylsulfonamide group include, but not limited to, acetylsulfamoyl [—S(═O)₂NHC(═O)Me], propionylsulfamoyl [—S(═O)₂NHC(═O)Et], isobutyrylsulfamoyl, butyrylsulfamoyl, and the like.

The term “C₁₋₄alkoxy” refers to an alkyl radical, as defined herein, attached directly to an oxygen atom (i.e., —O—C₁₋₄ alkyl). Examples include methoxy, ethoxy, n-propoxy, iso-propoxy, n-butoxy, t-butoxy, iso-butoxy, sec-butoxy and the like.

The term “C₁₋₄alkyl” refers to a straight or branched carbon radical containing 1 to 4 carbons, some embodiments are 1 to 3 carbons, some embodiments are 1 to 2 carbons. Examples of an alkyl include, but not limited to, methyl, ethyl, n-propyl, iso-propyl, n-butyl, iso-butyl, t-butyl, sec-butyl, and the like.

The term “C₁₋₄alkylamino” refers to one alkyl radical attached directly to an amino radical (—HN—C₁₋₄ alkyl) wherein the alkyl radical has the same meaning as described herein. Some examples include, but not limited to, methylamino (i.e., —HNCH₃), ethylamino, n-propylamino, iso-propylamino, n-butylamino, sec-butylamino, iso-butylamino, t-butylamino, and the like.

The term “C₁₋₄alkylcarboxamide” or “C₁₋₄alkylcarboxamido” refers to a single C₁₋₄alkyl group attached to the nitrogen of an amide group, wherein alkyl has the same definition as described herein. The C₁₋₄alkylcarboxamido may be represented by the following:

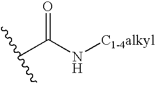

Examples include, but not limited to, N-methylcarboxamide, N-ethylcarboxamide, N-n-propylcarboxamide, N-iso-propylcarboxamide, N-n-butylcarboxamide, N-sec-butylcarboxamide, N-iso-butylcarboxamide, N-t-butylcarboxamide and the like.

The term “C₁₋₄alkylsulfonyl” refers to a alkyl radical attached to a sulfone radical of the formula: —S(O)₂— wherein the alkyl radical has the same definition as described herein. Examples include, but not limited to, methylsulfonyl, ethylsulfonyl, n-propylsulfonyl, iso-propylsulfonyl, n-butylsulfonyl, sec-butylsulfonyl, iso-butylsulfonyl, t-butyl, and the like.

The term “C₁₋₄alkylthio” refers to a alkyl radical attached to a sulfide of the formula: —S— wherein the alkyl radical has the same definition as described herein. Examples include, but not limited to, methylsulfanyl (i.e., CH₃S—), ethylsulfanyl, n-propylsulfanyl, iso-propylsulfanyl, n-butylsulfanyl, sec-butylsulfanyl, iso-butylsulfanyl, t-butyl, and the like.

The term “C₂₋₄alkynyl” refers to a radical containing 2 to 4 carbons and at least one carbon-carbon triple bond (—C≡C—), some embodiments are 2 to 3 carbons, and some embodiments have 2 carbons (—C≡CH). Examples of a C₂₋₄ alkynyl include, but not limited to, ethynyl, 1-propynyl, 2-propynyl, 1-butynyl, 2-butynyl, 3-butynyl, and the like. The term C₂₋₄ alkynyl includes di- and tri-ynes.

The term “amino” refers to the group —NH₂.

The term “carbo-C₁₋₆-alkoxy” refers to an alkoxy group attached directly to the carbon of a carbonyl and can be represented by the formula —C(═O)O—C₁₋₆-alkyl, wherein the C₁₋₆ alkyl group is as defined herein. In some embodiments, the carbo-C₁₋₆-alkoxy group is further bonded to a nitrogen atom and together form a carbamate group (e.g., NC(═O)O—C₁₋₆-alkyl). Examples of the carbo-C₁₋₆-alkoxy group include, but not limited to, methoxycarbonyl, ethoxycarbonyl, propoxycarbonyl, iso-propoxycarbonyl, butoxycarbonyl, sec-butoxycarbonyl, iso-butoxycarbonyl, t-butoxycarbonyl, n-pentoxycarbonyl, iso-pentoxycarbonyl, t-pentoxycarbonyl, neo-pentoxycarbonyl, n-hexyloxycarbonyl, and the like.

The term “cyano” refers to the group —CN.

The term “C₃₋₅cycloalkyl” refers to a saturated ring radical containing 3 to 5 carbons; some embodiments contain 3 to 4 carbons; some embodiments contain 3 carbons. Examples include cyclopropyl, cyclobutyl, cyclopentyl, and the like.

The term “C₃₋₅-cycloalkoxy” refers to a cycloalkyl, as defined herein, attached directly to an oxygen atom (i.e., —O—C₃₋₆cycloalkyl). Examples include, but not limited to, cyclopropoxy, cyclobutoxy, cyclopentoxy, and the like.

The term “di-C₁₋₄-dialkylamino” refers to an amino group substituted with two of the same or different C₁₋₄ alkyl radicals wherein alkyl radical has the same definition as described herein. Some examples include, but not limited to, dimethylamino, methylethylamino, diethylamino, methylpropylamino, methylisopropylamino, ethylpropylamino, ethylisopropylamino, dipropylamino, propylisopropylamino and the like.

The term “di-C₁₋₄-alkylcarboxamide” or “di-C₁₋₄-alkylcarboxamido” refers to two C₁₋₄ alkyl radicals, that are the same or different, attached to an amide group, wherein alkyl has the same definition as described herein. A di-C₁₋₄-alkylcarboxamido can be represented by the following group:

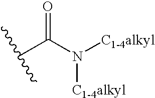

wherein C₁₋₄ has the same definition as described herein. Examples of a dialkylcarboxamide include, but not limited to, N,N-dimethylcarboxamide, N-methyl-N-ethylcarboxamide, N,N-diethylcarboxamide, N-methyl-N-isopropylcarboxamide, and the like.

The term “halogen” or “halo” refers to a fluoro, chloro, bromo or iodo group.

The term “heterocyclyl” refers to a non-aromatic carbon ring (i.e., cycloalkyl or cycloalkenyl) wherein one, two or three ring carbons are replaced by a heteroatom selected from, but not limited to, the group consisting of —O—, —S—, —S(═O)—, —S(═O)₂—, and —NH—, and the ring carbon atoms are optionally substituted with oxo or thiooxo thus forming a carbonyl or thiocarbonyl group respectively. The heterocyclic group can be a 3, 4, 5 or 6-member containing ring. Examples of a heterocyclic group, include but not limited to, aziridin-1-yl, aziridin-2-yl, azetidin-1-yl, azetidin-2-yl, azetidin-3-yl, piperidin-1-yl, piperidin-4-yl, morpholin-4-yl, piperzin-1-yl, piperzin-4-yl, pyrrolidin-1-yl, pyrrolidin-3-yl, [1,3]-dioxolan-2-yl and the like.

The term “hydroxyl” refers to the group —OH.

The term “oxadiazolyl” refers to the group represented by the following formulae:

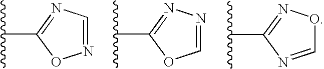

The term “oxo” refers generally to a double bonded oxygen; typically “oxo” is a substitution on a carbon and together form a carbonyl group.

The term “phosphonooxy” refers to a group of the formula —OP(O)(OH)₂ and can be represented by the following chemical structure:

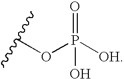

The term “pyrimidinyl” refers to the group represented by the following formulae:

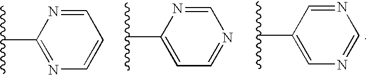

The term “sulfonamide” refers to the group —S(═O)₂NH₂.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

As used herein, the notation “*” shall denote the presence of a stereogenic center.

It is understood and appreciated that compounds of the invention may have one or more chiral centers, and therefore can exist as enantiomers and/or diastereomers. The invention is understood to extend to and embrace all such enantiomers, diastereomers and mixtures thereof, including, but not limited to, racemates. Accordingly, some embodiments of the present invention pertain to compounds that are R enantiomers. Further, some embodiments of the present invention pertain to compounds that are S enantiomers. When more than one chiral center is present, for example two chiral centers then, some embodiments of the present invention are compounds that are RS or SR enantiomers. In further embodiments, compounds of the present invention are RR or SS enantiomers. It is understood that compounds of Formula (Ia) and formulae used throughout this disclosure are intended to represent all individual enantiomers and mixtures thereof, unless stated or shown otherwise.

Compounds of the invention can also include tautomeric forms, such as keto-enol tautomers, and the like. Tautomeric forms can be in equilibrium or sterically locked into one form by appropriate substitution. It is understood that the various tautomeric forms are within the scope of the compounds of the present invention.

Compounds of the invention can also include all isotopes of atoms occurring in the intermediates and/or final compounds. Isotopes include those atoms having the same atomic number but different mass numbers. For example, isotopes of hydrogen include deuterium and tritium.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed in Journal of Pharmaceutical Science, 66, 2 (1977); incorporated herein by reference in its entirety.

The acid addition salts may be obtained as the direct product of compound synthesis. In the alternative, the free base may be dissolved in a suitable solvent containing the appropriate acid, and the salt isolated by evaporating the solvent or otherwise separating the salt and solvent. The compounds of this invention may form solvates with standard low molecular weight solvents using methods known to the skilled artisan.

In addition, compounds according to the invention may optionally exist as pharmaceutically acceptable basic addition salts. For example, these salts can be prepared in situ during the final isolation and purification of the compounds of the invention, or separately by reacting an acidic moiety, such as a carboxylic acid, with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, “Pro-drugs as Novel Delivery Systems,” Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

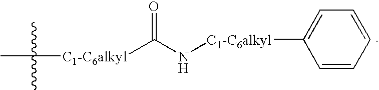

Abbreviations used in the specification, particularly the Schemes and Examples, are as follows

BMI=Body Mass Index

DMF=N,N-Dimethylformamide

DMSO=Dimethylsulfoxide

EOD=Early-Onset Type 2 Diabetes

EtOAc=Ethyl acetate

HPLC=High Pressure Liquid Chromatography

IDDM=Insulin Dependent Diabetes Mellitus

IGT=Impaired Glucose Tolerance

IPA=Isopropyl Alcohol

KO-t-Bu=Potassium tert-Butoxide

LADA=Latent Autoimmune Diabetes in Adults

Me=Methyl

2-Me-THF=2-Methyl-tetrahydrofuran

MODY=Maturity Onset Diabetes of the Young

MOM=Methoxymethyl ether

MTBE=Methyl t-Butyl Ether

NIDDM=Non-Insulin Dependent Diabetes Mellitus

NMP=N-methyl-2-pyrrolidinone

TEA=Triethylamine

THF=Tetrahydrofuran

THP=Tetrahydropyran

TMEDA=Tetramethylethylenediamine

TMPDA=Tetramethylpropylenediamine

TMS=Trimethylsilyl

YOAD=Youth-Onset Atypical Diabetes

As used herein, unless otherwise noted, the term “isolated form” shall mean that the compound is present in a form which is separate from any solid mixture with another compound(s), solvent system or biological environment. In an embodiment of the present invention, the compound of formula (I) is prepared as an isolated form. In another embodiment of the present invention, the compound of formula (I-S) is prepared as an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is prepared as a substantially pure form. In another embodiment of the present invention, the compound of formula (I-S) is prepared as a substantially pure form.

As used herein, unless otherwise noted, the term “substantially free of a corresponding salt form(s)” when used to described the compound of formula (I) shall mean that mole percent of the corresponding salt form(s) in the isolated compound of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt forms. In another embodiment of the present invention, the compound of formula (I-S) is present in a form which is substantially free of corresponding salt forms.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

Examples of suitable solvents, bases, reaction temperatures, and other reaction parameters and components are provided in the detailed descriptions which follows herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

For example, terminal amino or alkylamino groups may be protected with a suitably selected nitrogen protecting group. As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R' is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

In another example, terminal hydroxy groups may be protected with a suitably selected oxygen protecting group. As used herein, unless otherwise noted, the term “oxygen protecting group” shall mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention is directed to processes for the preparation of compounds of formula (I) as outlined in more detail in Scheme 1 below.

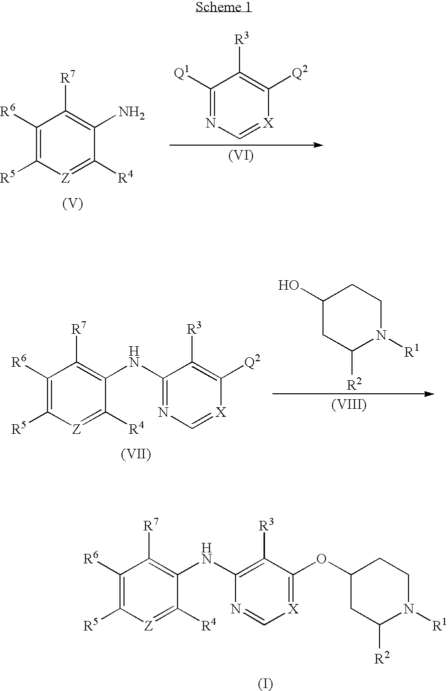

Accordingly, a suitably substituted compound of formula (V), a known compound or compound prepared by known methods, is reacted with a suitably substituted compound of formula (VI), wherein Q¹ and Q² are each independently a suitably selected leaving group such as chloro, fluoro, bromo, methanesulfonate, and the like, preferably Q¹ and Q² are the same, more preferably Q¹ and Q² are the same and are each chloro; a known compound or compound prepared by known methods; wherein the compound of formula (VI) is preferably present in an amount in the range of from about 1.0 to about 5.0 molar equivalents, more preferably about 3.0 molar equivalents;

in the presence of a suitably selected acid catalyst such as HCl, H₂SO₄, CH₃SO₃H, and the like, preferably CH₃SO₃H; wherein the acid catalyst is preferably present in an amount in the range of from about 1.0 to about 5.0 molar equivalents, more preferably about 2.0 molar equivalents; in a first organic solvent such as acetonitrile, IPA, CH₃OCH₂CH₂OH, and the like, preferably acetonitrile; preferably at a temperature in the range of from about 60° C. to about 80° C., more preferably at about 75° C.; to yield the corresponding compound of formula (VII).

Alternatively, a suitably substituted compound of formula (V), a known compound or compound prepared by known methods, is reacted with a suitably substituted compound of formula (VI), wherein Q¹ and Q² are each independently a suitably selected leaving group such as chloro, fluoro, bromo, methanesulfonate, and the like, preferably Q¹ and Q² are the same, more preferably Q¹ and Q² are the same and are each chloro, a known compound or compound prepared by known methods; wherein the compound of formula (VI) is preferably present in an amount in the range of from about 1.0 to about 3.0 molar equivalents, more preferably, in an amount in the range of from about 1.0 to about 1.5 molar equivalents, more preferably in an amount of about 1.3 molar equivalents;

in the presence of a suitably selected carbonate base such as Cs₂CO₃, K₂CO₃, Na₂CO₃, and the like, preferably Cs₂CO₃; wherein the carbonate base is preferably present in an amount in the range of from about 1.0 to about 3.0 molar equivalents, more preferably about 1.5 molar equivalents; in a second organic solvent such as DMF, DMSO, NMP, and the like, preferably DMSO; preferably at a temperature in the range of from about 50° C. to about 90° C., more preferably at about 60° C.; to yield the corresponding compound of formula (VII).

The compound of formula (VII) is reacted with a suitably substituted compound of formula (VIII), a known compound or compound prepared by known methods; wherein the compound of formula (VIII) is preferably present in an amount in the range of from about 1.0 to about 20.0 molar equivalents, more preferably in an amount in the range of from about 5.0 to about 15.0 molar equivalents, more preferably about 10.0 molar equivalents;

in the presence of a suitably selected base, which base is strong enough to de-protonate the hydroxy group which is bound at the 4-position of the piperidinyl portion of the compound of formula (VIII), preferably an inorganic base such as potassium t-butoxide, NaH, KH, and the like, more preferably potassium t-butoxide; wherein the base is preferably present in an amount in the range of from about 1.0 to about 10.0 molar equivalents, more preferably in an amount in the range of from about 2.0 to about 5.0 molar equivalents, more preferably in an amount of about 3.0 molar equivalents; in a third organic solvent or mixture of organic solvents such as 1,4-dioxane, 2-methyl-THF, THF, and the like, preferably 1,4-dioxane; optionally in the presence of an additive such as TEA, pyridine, TMEDA, TMPDA, and the like, preferably TMPDA; preferably at a temperature in the range of from about 60° C. to about 90° C., more preferably at a temperature of about 85° C.; to yield the corresponding compound of formula (I).

One skilled in the art will recognize that wherein the compound of formula (I), one or more of the R¹, R², R³, R⁴, R⁵, R⁶ and/or R⁷ substituent groups contains a terminal hydroxy (—OH), amino (—NH₂) or alkylamino (—NH(alkyl)) group, said group is preferably protected prior to the reaction of the compound of formula (V) with the compound of formula (VI), in the presence of the suitably selected carbonate base, to yield the corresponding compound of formula (VII). The protecting group may then be removed either following the preparation of the compound of formula (VII) or following the reaction of the corresponding protected compound of formula (VII) with a suitably substituted compound of formula (VIII) to yield the corresponding protected compound of formula (I).

In an embodiment, the present invention is directed to a process for the preparation of a compound of formula (I-S) as described in more detail in Scheme 2, below.

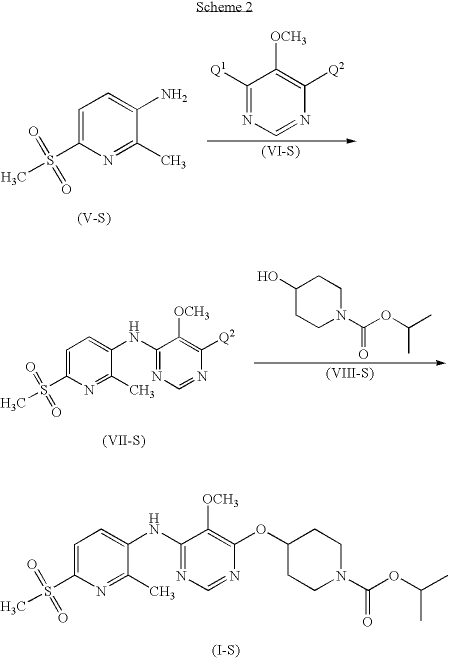

Accordingly, a suitably substituted compound of formula (V-S), a known compound or compound prepared by known methods, is reacted with a suitably substituted compound of formula (VI-S), wherein Q¹ and Q² are each independently a suitably selected leaving group such as chloro, fluoro, bromo, methanesulfonate, and the like, preferably Q¹ and Q² are the same, more preferably Q¹ and Q² are the same and are each chloro; a known compound or compound prepared by known methods; wherein the compound of formula (VI-S) is preferably present in an amount in the range of from about 1.0 to about 5.0 molar equivalents, more preferably about 3.0 molar equivalents;

in the presence of a suitably selected acid catalyst such as HCl, H₂SO₄, CH₃SO₃H, and the like, preferably CH₃SO₃H; wherein the acid catalyst is preferably present in an amount in the range of from about 1.0 to about 5.0 molar equivalents, more preferably about 2.0 molar equivalents; in a first organic solvent such as acetonitrile, IPA, CH₃OCH₂CH₂OH, and the like, preferably acetonitrile; preferably at a temperature in the range of from about 60° C. to about 80° C., more preferably at about 75° C.; to yield the corresponding compound of formula (VII-S).

Alternatively, a suitably substituted compound of formula (V-S), a known compound or compound prepared by known methods, is reacted with a suitably substituted compound of formula (VI-S), wherein Q¹ and Q² are each independently a suitably selected leaving group such as chloro, fluoro, bromo, methanesulfonate, and the like, preferably Q¹ and Q² are the same, more preferably Q¹ and Q² are the same and are each chloro, a known compound or compound prepared by known methods; wherein the compound of formula (VI) is preferably present in an amount in the range of from about 1.0 to about 3.0 molar equivalents, more preferably, in an amount in the range of from about 1.0 to about 1.5 molar equivalents, ore preferably in an amount of about 1.3 molar equivalents;

in the presence of a suitably selected carbonate base such as Cs₂CO₃, K₂CO₃, Na₂CO₃, and the like, preferably Cs₂CO₃; wherein the carbonate base is preferably present in an amount in the range of from about 1.0 to about 3.0 molar equivalents, more preferably about 1.5 molar equivalents; in a second organic solvent such as DMF, DMSO, NMP, and the like, preferably DMSO; preferably at a temperature in the range of from about 50° C. to about 90° C., more preferably at about 60° C.; to yield the corresponding compound of formula (VII-S).

The compound of formula (VII-S) is reacted with a suitably substituted compound of formula (VIII-S), a known compound or compound prepared by known methods; wherein the compound of formula (VIII) is preferably present in an amount in the range of from about 1.0 to about 20.0 molar equivalents, more preferably in an amount in the range of from about 5.0 to about 15.0 molar equivalents, more preferably about 10.0 molar equivalents;

in the presence of a suitably selected base, which base is strong enough to de-protonate the hydroxy group which is bound at the 4-position of the piperidinyl portion of the compound of formula (VIII), preferably an inorganic base such as potassium t-butoxide, NaH, KH, and the like, more preferably potassium t-butoxide; wherein the base is preferably present in an amount in the range of from about 1.0 to about 10.0 molar equivalents, more preferably in an amount in the range of from about 2.0 to about 5.0 molar equivalents, more preferably in an amount of about 3.0 molar equivalents; in a third organic solvent or mixture of organic solvents such as 1,4-dioxane, 2-methyl-THF, THF, and the like, preferably 1,4-dioxane; optionally in the presence of an additive such as TEA, pyridine, TMEDA, TMPDA, and the like, preferably TMPDA; preferably at a temperature in the range of from about 60° C. to about 90° C., more preferably at a temperature of about 85° C.; to yield the corresponding compound of formula (I-S).

The present invention further comprises pharmaceutical compositions containing a compound prepared according to any of the processes described herein (more preferably a compound of formula (I-S) prepared according to any of the processes described herein) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01-1000 mg or any range therein, and may be given at a dosage of from about 0.01-500 mg/kg/day, or any range therein, preferably from about 0.5-300 mg/kg/day, or any range therein, more preferably from about 1.0-100 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 to about 1000 mg, or any range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating metabolic related disorders described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 1.0 to 500 mg of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of metabolic related disorders is required.

The daily dosage of the products may be varied over a wide range from 0.01 to 5,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1,000 milligrams of the active ingredient for the symptomatic adjustment of the dosage to the patient to be treated. An effective amount of the drug is ordinarily supplied at a dosage level of from about 0.01 mg/kg to about 500 mg/kg of body weight per day, or any range therein. Preferably, the range is from about 0.01 to about 300.0 mg/kg of body weight per day, or any range therein. More preferably, from about 0.5 to about 300.0 mg/kg of body weight per day, or any range therein. More preferably, from about 1.0 to about 100.0 mg/kg of body weight per day, or any range therein. More preferably, from about 10.0 to about 50.0 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter. In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### (6-Chloro-5-methoxy-pyrimidin-4-yl)-(6-methanesulfonyl-2-methyl-pyridin-3-yl)-amine

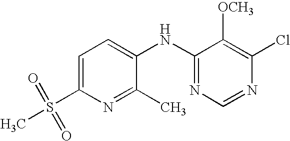

A 100-mL Schlenk flask with a magnetic stir bar was charged with 4,6-dichloro-5-methoxypyrimidine (4.66 g, 0.026 mol), 6-methanesulfonyl-2-methyl-3-pyridinamine (3.72 g, 0.020 mol), and DMSO (20 mL). After the mixture was stirred to dissolve the components, Cs₂CO₃ (8.48 g, 0.026 mol) was added, and the resulting mixture heated to 60° C. After 4.5 h and additional portion of Cs₂CO₃ (2.00 g, 0.0061 mol) was added and heating continued overnight. The reaction was quenched by addition of the reaction mixture to well stirred, saturated NH₄Cl (200 mL); resulting in the formation of a tan precipitate. The precipitate was filtered, air dried and stirred with MTBE, and the resulting mixture filtered again. The resulting solid was purified by column chromatography on silica gel (200 g) using CH₂Cl₂ followed by 1% IPA-CH₂Cl₂ after 2-L of CH₂Cl₂ had eluted to yield the title compound as a white to light yellow solid.

¹H NMR (300 MHz, CDCl₃) δ: 8.99 (d, 1H, J=8.6 Hz), 8.37 (s, 1H), 8.00 (d, 1H, J=8.6 Hz), 7.49 (br s, 1H), 4.08 (s, 3H), 3.21 (s, 3H), 2.69 (s, 3H).

HRMS: [MH]⁺ m/z=329.0465.

NOTE: (6-Chloro-5-methoxy-pyrimidin-4-yl)-(6-methanesulfonyl-2-methyl-pyridin-3-yl)-methyl-amine was also isolated as a by-product from the above reaction mixture.

### Example 2

### 4-[6-(6-Methanesulfonyl-2-methyl-pyridin-3-ylamino)-5-methoxy-pyrimidin-4-yloxy]-piperidine-1-carboxylic acid isopropyl ester

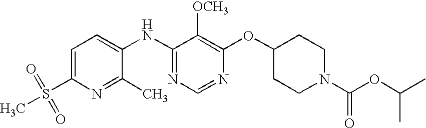

A 100 mL Schlenk flask with a magnetic stir bar, septum, heating mantle, and thermocouple was charged with (6-chloro-5-methoxy-pyrimidin-4-yl)-(6-methanesulfonyl-2-methyl-pyridin-3-yl)-amine (658.1 mg, 2 mmol), blocked 4-hydroxypiperidine (3.753 g, 20 mmol), 1,4-dioxane (20 mL) followed by KO-t-Bu (455 mg, 4 mmol). On addition of KO-t-Bu a pronounced exotherm was observed. After the exotherm had subsided, the he resulting mixture was heated to 85° C. and followed by HPLC. When the reaction progress began to slow, an additional charge of KO-t-Bu (230 mg, 2.05 mmol) was added (Note 2). When the reaction progress began to slow again, a third charge of KO t-Bu (110 mg, 1 mmol) was added. The reaction mixture was quenched by pouring the reaction into well-stirred, saturated NH₄Cl (250 mL), then extracted with EtOAc (2×50 mL), and concentration to yield an oil (3.63 g). The oil was taken up in MTBE and filtered through CELITE® to remove insoluble components. The resulting solution (approximately 25 mL) was treated with heptane (100 mL), and the resulting mixture stirred rapidly until the formation of a solid was observed (30 min). The solid was filtered to yield the title compound. The solid product was recrystallized from EtOAc-heptane (1:2) to yield the title compound as a solid.

¹H NMR (300 MHz, CDCl₃) δ: 9.02 (d, 1H, J=8.6 Hz), 8.20 (s, 1H), 7.97 (d, 1H, J=8.6 Hz), 7.31 (br s, 1H), 5.40 (m, 1H), 4.94 (sept, 1H, J=6.2 Hz), 4.00 (s, 3H), 3.80 (m, 2H), 3.41 (m, 2H), 3.19 (s, 3H), 2.66 (s, 3H), 2.04 (m, 2H), 1.83 (m, 2H), 1.26 (d, 6H, J=6.2 Hz).

LCMS: [MH]⁺ m/z 480, [M+23]⁺ m/z 502.

### Example 3

### 6-Chloro-5-methoxy-N-methyl-N-(2-methyl-6-(methylsulfonyl)pyridin-3-yl)pyrimidin-4-amine

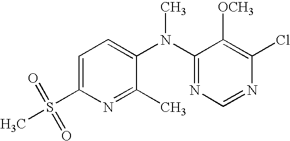

A 50 mL single neck round-bottom flask with a magnetic stir bar, septum and thermocouple was charged with 6-chloro-5-methoxy-N-(2-methyl-6-(methylsulfonyl)pyridin-3-yl)pyrimidin-4-amine (332.64 mg, 1.01 mmol), DMF (10 mL), Cs₂CO₃ (378.2 mg, 1.16 mmol), and methyl iodide (180.5 mg, 1.27 mmol). The reaction was allowed to stir at room temperature for approximately 48 h. The resulting mixture was then diluted with an equal volume of water, extracted with MTBE (3×15 mL), and concentrated to yield the title compound as a light yellow solid.

¹H NMR (300 MHz, CDCl₃) δ: 8.34 (s, 1H), 8.00 (d, J=8.1 Hz, 1H), 7.70 (d, J=8.1 Hz, 1H), 3.47 (s, 3H), 3.28 (s, 1H), 3.24 (s, 3H).

### Example 4

### 4-[6-(6-Methanesulfonyl-2-methyl-pyridin-3-ylamino)-5-methoxy-pyrimidin-4-yloxy]-piperidine-1-carboxylic acid isopropyl ester

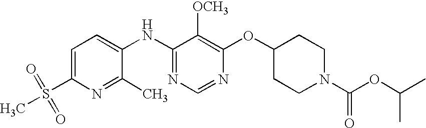

A 5 mL vial with a magnetic stir bar was charged with the (6-chloro-5-methoxy-pyrimidin-4-yl)-(6-methanesulfonyl-2-methyl-pyridin-3-yl)-amine (32.9 mg, 0.10 mmol), isopropyl 4-hydroxypiperidine-1-carboxylate (189.3 mg, 1.01 mmol), THF (1 mL), tetramethylpropylenediamine (47.6 mg, 0.37 mmol), and potassium t-butoxide (38.5 mg, 0.315 mmol). The resulting mixture was heated at 70° C. and followed by HPLC to check the reaction progress. After 30 h HPLC indicated formation of the title compound (30%).

### Example 5

### 6-Chloro-5-methoxy-pyrimidin-4-yl)-(6-methanesulfonyl-2-methyl-pyridin-3-yl)-amine

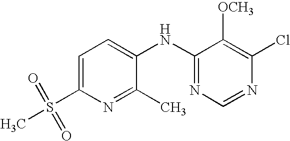

A 100 mL 3 neck flask equipped with a magnetic stir bar, thermocouple, and condenser was charged with 4,6-dichloro-5-methoxypyrimidine (3.39 g, 18.9 mmol), 6-methanesulfonyl-2-methyl-3-pyridinamine (2.34 g, 12.6 mmol), acetonitrile (20 mL), and freshly prepared 1M methanesulfonic acid in water (35 mL, 35 mmol). The resulting mixture was heated to 85° C. for 18 h. To the resulting mixture was then added an additional portion of 4,6-dichloro-5-methoxypyrimidine (3.39 g, 18.9 mmol) and heating continued for an additional 18 h. The resulting mixture was allowed to cool to room temperature, the solid mass was filtered and washed with chilled acetonitrile (20 mL) to yield as a white solid. The white solid was recrystallized from acetonitrile (350 mL) to yield a first crop of unreacted 6-methanesulfonyl-2-methyl-3-pyridinamine as a white solid. Cooling of the filtrate yielded a second crop of the title compound as a white solid. The filtrate was then concentrated and the resulting solid was partitioned between ethyl acetate and water, the water layer was extracted with dichloromethane to yield an amount of the title compound. Finally, the water layer was concentrated to yield yet another amount of the title compound as a while solid.

¹H NMR (300 MHz, CDCl₃) δ: 9.00 (d, 1H, J=8.6 Hz), 8.38 (s, 1H), 8.01 (d, 1H, J=8.6 Hz), 7.48 (br s, 1H), 4.09 (s, 3H), 3.21 (s, 3H), 2.69 (s, 3H).

LCMS: [MH]⁺ m/z=329, [2M+23]⁺ m/z=679.

NOTE: 6-Chloro-4-hydroxy-5-methoxypyrimidine was prepared as a by-product of the above reaction (from hydrolysis of the 4,6-dichloro-5-methoxypyrimidine under the acidic reaction conditions).

### Example 6

### Oral Formulation—Prophetic Example

As a specific embodiment of an oral composition, 100 mg of the compound prepared as in Example 2 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of a compound of formula (I) 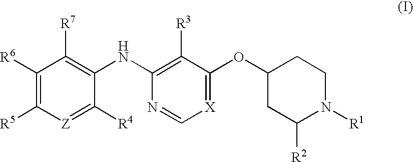
wherein:
X is N or CR⁸; wherein R⁸ is H or halogen;
Z is CH or N;
R¹ is carbo-C₁₋₆-alkoxy, oxadiazolyl or pyrimidinyl wherein said carbo-C₁₋₆-alkoxy, oxadiazolyl and pyrimidinyl are each optionally substituted with 1 or 2 substituents selected independently from the group consisting of C₁₋₄alkyl, C₁₋₄alkoxy and C₃₋₅cycloalkyl;
R² is H or C₁₋₄alkyl;
R³ is C₁₋₄ alkoxy, O—C₂₋₄-alkynyl or hydroxyl;
R⁴ is selected from the group consisting of H, C₁₋₄alkoxy, C₁₋₄alkyl, C₂₋₄alkynyl and halogen;
R⁵ is selected from the group consisting of C₁₋₄acylsulfonamide, C₁₋₄alkoxy, C₁₋₄alkyl, C₁₋₄alkylamino, C₁₋₄alkylsulfonyl, C₁₋₄alkylthio, cyano, heterocyclyl, di-C₁₋₄-dialkylamino and sulfonamide, wherein said C₁₋₄alkoxy, C₁₋₄alkyl, C₁₋₄alkylamino, C₁₋₄alkylsulfonyl, C₁₋₄alkylthio, di-C₁₋₄-dialkylamino and heterocyclyl are each optionally substituted with 1 or 2 substituents selected independently from the group consisting of C₂₋₄alkynyl, C₁₋₄alkoxy, C₁₋₄alkylcarboxamide, C₁₋₄alkylsulfonyl, C₃₋₅cycloalkyl, C₃₋₅cycloalkyloxy, di-C₁₋₄-alkylcarboxamide, hydroxyl and phosphonooxy, wherein said C₁₋₄alkylcarboxamide is optionally substituted with hydroxyl; or
or R⁵ is a group of Formula (A): 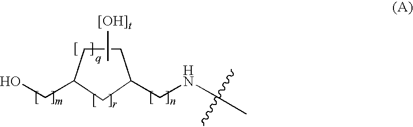
wherein “m”, “n” and “q” are each independently 0, 1, 2 or 3; “r” is 0, 1 or 2; and “t” is 0 or 1;
R⁶ is H or halogen;
R⁷ is H or C₁₋₄alkyl;
or a pharmaceutically acceptable salt, solvate or hydrate thereof;
comprising 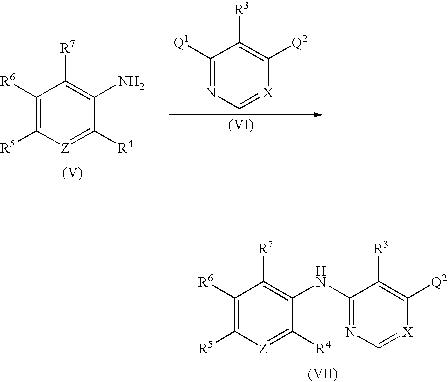
reacting a compound of formula (V) with a compound of formula (VI), wherein Q¹ and Q² are each independently a leaving group; in the presence of carbonate base; in a second organic solvent; to yield the corresponding compound of formula (VII); 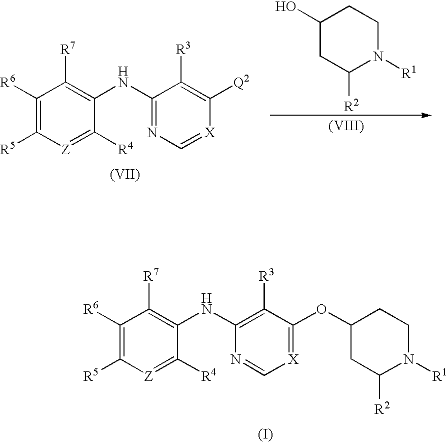
reacting the compound of formula (VII) with a compound of formula (VIII); in the presence of a base, which base is strong enough to de-protonate the hydroxy group which is bound at the 4-position of the piperidinyl portion of the compound of formula (VIII); in a third organic solvent; to yield the corresponding compound of formula (I).

2. A process as in claim 1, wherein Q¹ and Q² are the same and are each chloro.

3. A process as in claim 1, wherein the compound of formula (VI) is present in an amount in the range of from about 1.0 to about 3.0 molar equivalents.

4. A process as in claim 1, wherein the carbonate base is selected from the group consisting of Cs₂CO₃, K₂CO₃ and Na₂CO₃.

5. A process as in claim 1, wherein the carbonate base is present in an amount in the range of from about 1.0 to about 3.0 molar equivalents.

6. A process as in claim 1, wherein the carbonate base is Cs₂CO₃ and wherein the carbonate base is present in an amount of about 1.5 equivalents.

7. A process as in claim 1, wherein the second organic solvent is selected from the group consisting of DMF, DMSO and NMP.

8. A process as in claim 7, wherein the second organic solvent is DMF.

9. A process as in claim 1, wherein the compound of formula (V) is reacted with the compound of formula (VI) at a temperature in the range of from about 50° C. to about 90° C.

10. A process as in claim 1, wherein the compound of formula (VIII) is present in an amount in the range of from about 5.0 to about 15.0 molar equivalents.

11. A process as in claim 1, wherein the base which is strong enough to de-protonate the hydroxy group which is bound at the 4-position of the piperidinyl portion of the compound of formula (VIII) is selected from the group consisting of potassium t-butoxide, NaH and KH.

12. A process as in claim 1, wherein the base which is strong enough to de-protonate the hydroxy group which is bound at the 4-position of the piperidinyl portion of the compound of formula (VIII) is present in an amount in the range of from about 2.0 to about 5.0 molar equivalents.

13. A process as in claim 1, wherein the base which is strong enough to de-protonate the hydroxy group which is bound at the 4-position of the piperidinyl portion of the compound of formula (VIII) is potassium t-butoxide and wherein the base is present in an amount of about 3.0 molar equivalents.

14. A process as in claim 1, wherein the third organic solvent is selected from the group consisting of 1,4-dioxane, THF and 2-methyl-THF.

15. A process as in claim 14, wherein the third organic solvent is 1,4-dioxane.

16. A process as in claim 1, wherein the compound of formula (VII) is reacted with the compound of formula (VIII) at a temperature in the range of from about 60° C. to about 90° C.

17. A process as in claim 1, wherein the compound of formula (VII) is reacted with the compound of formula (VIII) in the presence of an additive.

18. A process as in claim 17, wherein the additive is selected from the group consisting of TEA, pyridine, TMEDA and TMPDA.

19. A process as in claim 18, wherein the additive is TMPDA.

20. A compound prepared according to a process as in claim 1.

21. A process for the preparation of a compound of formula (I-S) 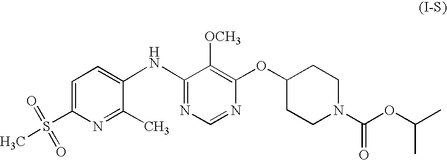
or a pharmaceutically acceptable salt, solvate or hydrate thereof;
comprising 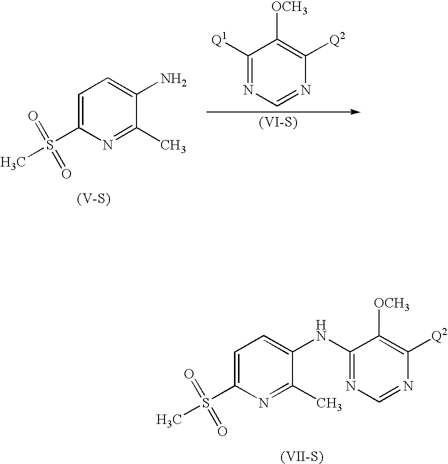
reacting a compound of formula (V-S) with a compound of formula (VI-S), wherein Q¹ and Q² are each independently a leaving group; in the presence of carbonate base; in a second organic solvent; to yield the corresponding compound of formula (VII-S); 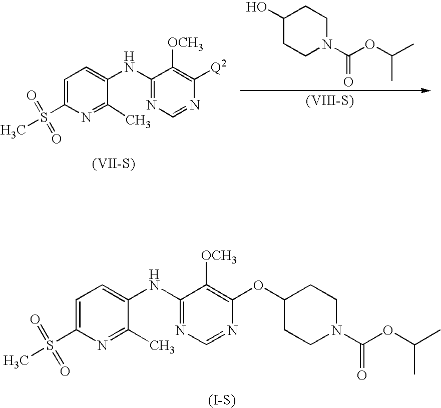
reacting the compound of formula (VII-S) with a compound of formula (VIII-S); in the presence of a base, which base is strong enough to de-protonate the hydroxy group which is bound at the 4-position of the piperidinyl portion of the compound of formula (VIII-S); in a third organic solvent; to yield the corresponding compound of formula (I-S).

22. A process as in claim 21, wherein Q¹ and Q² are the same and are each chloro.

23. A process as in claim 21, wherein the compound of formula (VI-S) is present in an amount in the range of from about 1.0 to about 3.0 molar equivalents.

24. A process as in claim 21, wherein the carbonate base is selected from the group consisting of Cs₂CO₃, K₂CO₃ and Na₂CO₃.

25. A process as in claim 21, wherein the carbonate base is present in an amount in the range of from about 1.0 to about 3.0 molar equivalents.

26. A process as in claim 21, wherein the carbonate base is Cs₂CO₃ and wherein the carbonate base is present in an amount of about 1.5 equivalents.

27. A process as in claim 21, wherein the second organic solvent is selected from the group consisting of DMF, DMSO and NMP.

28. A process as in claim 27, wherein the second organic solvent is DMF.

29. A process as in claim 21, wherein the compound of formula (V-S) is reacted with the compound of formula (VI-S) at a temperature in the range of from about 50° C. to about 90° C.

30. A process as in claim 21, wherein the compound of formula (VIII-S) is present in an amount in the range of from about 5.0 to about 15.0 molar equivalents.

31. A process as in claim 21, wherein the base which is strong enough to de-protonate the hydroxy group which is bound at the 4-position of the piperidinyl portion of the compound of formula (VIII-S) is selected from the group consisting of potassium t-butoxide, NaH and KH.

32. A process as in claim 21, wherein the base which is strong enough to de-protonate the hydroxy group which is bound at the 4-position of the piperidinyl portion of the compound of formula (VIII-S) is present in an amount in the range of from about 2.0 to about 5.0 molar equivalents.

33. A process as in claim 21, wherein the base which is strong enough to de-protonate the hydroxy group which is bound at the 4-position of the piperidinyl portion of the compound of formula (VIII-S) is potassium t-butoxide and wherein the base is present in an amount of about 3.0 molar equivalents.

34. A process as in claim 21, wherein the third organic solvent is selected from the group consisting of 1,4-dioxane, THF and 2-methyl-THF.

35. A process as in claim 34, wherein the third organic solvent is 1,4-dioxane.

36. A process as in claim 21, wherein the compound of formula (VII-S) is reacted with the compound of formula (VIII-S) at a temperature in the range of from about 60° C. to about 90° C.

37. A process as in claim 21, wherein the compound of formula (VII-S) is reacted with the compound of formula (VIII-S) in the presence of an additive.

38. A process as in claim 37, wherein the additive is selected from the group consisting of TEA, pyridine, TMEDA and TMPDA.

39. A process as in claim 38, wherein the additive is TMPDA.

40. A compound prepared according to a process as in claim 21.

41. A pharmaceutical composition comprising a pharmaceutically acceptable carrier and a compound prepared as in claim 21.

42. A pharmaceutical composition made by mixing a compound prepared as in claim 21 and a pharmaceutically acceptable carrier.

43. A process for making a pharmaceutical composition comprising mixing a compound prepared as in claim 21 and a pharmaceutically acceptable carrier.

44. A method of treating a metabolic related disorder comprising administering to a subject a need thereof a therapeutically effective amount of the compound prepared as in claim 21.

45. The method of claim 44, wherein the metabolic related disorder is selected from the group consisting of hyperlipidemia, type 1 diabetes, type 2 diabetes mellitus, idiopathic type 1 diabetes (Type 1 b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction, dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin disorders, connective tissue disorders, foot ulcerations, ulcerative colitis, endothelial dysfunction and impaired vascular compliance.

46. The method of claim 44, wherein the metabolic related disorder is selected from the group consisting of type I diabetes, type II diabetes, inadequate glucose tolerance, insulin resistance, hyperglycemia, hyperlipidemia, hypertriglyceridemia, hypercholesterolemia, dyslipidemia and syndrome X.

47. The method of claim 44, wherein the metabolic related disorder is obesity.

48. A method of treating a metabolic related disorder selected from the group consisting of type I diabetes, type II diabetes, inadequate glucose tolerance, insulin resistance, hyperglycemia, hyperlipidemia, hypertriglyceridemia, hypercholesterolemia, dyslipidemia, syndrome X and obesity, comprising administering to a subject in need thereof a therapeutically effective amount of a compound prepared as in claim 21.

49. The use of a compound prepared as in claim 21 for the preparation of a medicament for the treatment of a metabolic related disorder in a subject in need thereof.
